SEQUENCE LISTING (11 sequences)

SEQ ID NO: 1 - DNA, 19 bp, synthetic construct
  source 1..19: mol_type = other DNA
gtgaggagattcacaagga

SEQ ID NO: 2 - DNA, 19 bp, synthetic construct
  source 1..19: mol_type = other DNA
gtcctgtacttcagcatct

SEQ ID NO: 3 - DNA, 19 bp, synthetic construct
  source 1..19: mol_type = other DNA
gcacaatctttacctcaga

SEQ ID NO: 4 - DNA, 20 bp, synthetic construct
  source 1..20: mol_type = other DNA
taacggaaaagggccgcgcc

SEQ ID NO: 5 - protein, 30 aa, synthetic construct
KFFQYDTWKQSTQRLRRGLPALLRARRGHC

SEQ ID NO: 6 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 7 - protein, 180 aa, Mus sp.
MGIPVGKSMLVLLISLAFALCCIAAYGPGETLCGGELVDTLQFVCSDRGFYFSRPSSRANRRSRGIVEECCFRSCDLALLETYCATPAKSERDVSTSQAVLPDDFPRYPVGKFFQYDTWRQSAGRLRRGLPALLRARRGRMLAKELKEFREAKRHRPLIVLPPKDPAHGGASSEMSSNHQ

SEQ ID NO: 8 - protein, 179 aa, Ovis sp.
MGITAGKSMLALLAFLAFASCCYAAYRPSETLCGGELVDTLQFVCGDRGFYFSRPSSRINRRSRGIVEECCFRSCDLALLETYCAAPAKSERDVSASTTVLPDDFTAYPVGKFFQSDTWKQSTQRLRRGLPAFLRARRGRTLAKELEALREAKSHRPLIALPTQDPATHGGASSEASSD

SEQ ID NO: 9 - protein, 186 aa, Gallus sp.
MCAARQILLLLLAFLAYALDSAAAYGTAETLCGGELVDTLQFVCGDRGFYFSRPVGRNNRRINRGIVEECCFRSCDLALLETYCAKSVKSERDLSATSLAGLPALNKESFQKPSHAKYSKYNVWQKKSSQRLQREVPGILRARRYWQAEGLQAAEEARAMHRPLISLPSQRPPAPRASPEATGPQE

SEQ ID NO: 10 - protein, 217 aa, Xenopus sp.
MEQLSCKHRSSSVEAEAQLCRQTESRSTQLPRMSVMRHLFLLSITFLVYTLDSAKAYRATETLCGGELVDTLQFVCGDRGFYFSTNNGRSNRRPNRGIVDVCCFKSCDLELLETYCAKPTKNERDVSTAPATAIPPLSKQDLYHKHHHTKSSKYDIWQRKSIHRLRRGVPAIVRARQYRLLMEKAEEAEQALSHRPLTTLPITRPLRLQQASEPSHN

SEQ ID NO: 11 - protein, 197 aa, Danio rerio
MDDYHVFCASCRKTEETRTTMRSLIVFVLSLSMLISNVTAGETLCGGELVDTLQFVCGEDGFYISRPNRSNSRRPQRGIVEECCFRSCELHLLQQYCAKPVKSERDVSSTSLQVFPVSQALHKDTINVKYSKYEVWQQKAAQRLRRGVPSILLARKFRRQMEKIQDEEQTSFHRPLMTLPNRQPAIVPHVQISTSRK

(19) United States
(12) Patent Application Publication
(10) Pub. No.: US 2025/0144242 A1
(43) Pub. Date: May 8, 2025

(54) DISRUPTION OF THE SECRETION OF INSULIN-LIKE GROWTH FACTOR 2 FOR CANCER TREATMENT

(71) Applicant: The Hong Kong University of Science and Technology, Hong Kong (CN)

(72) Inventors: Yusong GUO, Hong Kong (CN); Tiantian LI, Hong Kong (CN)

(21) Appl. No.: 18/932,949

(22) Filed: Oct. 31, 2024

Related U.S. Application Data
(60) Provisional application No. 63/595,766, filed on Nov. 3, 2023.

(51) Int. Cl. A61K 48/00; A61K 38/17; A61K 38/30; C12N 9/22; C12N 15/11

(52) U.S. Cl. CPC A61K 48/005; A61K 38/1709; A61K 38/30; C12N 9/22; C12N 15/111; C12N 2310/20

(57) ABSTRACT

The subject invention pertains to compositions and methods for inhibiting IGF2 signaling. Insulin-like growth factor 2 (IGF2) is a key signaling molecule that plays important roles in various physiological processes, including skeletal myogenesis during development and adult muscle remodeling. Abnormal activation of the signaling pathway induced by IGF2 has been shown to promote cancer progression. TMED10, a p24 family protein, functions as a cargo receptor, promoting the export of IGF2 from the endoplasmic reticulum (ER) via recognizing an ER export signal on IGF2. Moreover, TMED10 also mediates ER export of sortilin, which is important to regulate the export of IGF2 from the trans-Golgi network. The subject invention features a novel therapeutic strategy for cancer treatment, that includes, but is not limited to, inhibitors targeting the interaction between IGF2 and TMED10.

### CROSS-REFERENCE TO RELATED APPLICATION

The present application claims the benefit of U.S. Provisional Application Ser. No. 63/595,766, filed Nov. 3, 2023, which is hereby incorporated by reference herein in its entirety, including any figures, tables, or drawings.

### SEQUENCE LISTING

The Sequence Listing for this application is labeled “HKUS-197X-SeqList.xml” which was created on Oct. 16, 2024 and is 11,619 bytes. The entire contents of the sequence listing is incorporated herein by reference in its entirety.

### BACKGROUND OF THE INVENTION

Insulin-like growth factor 2 (IGF2) is a pivotal player in various cellular processes such as proliferation, migration, differentiation, and survival. IGF2 is involved in various physiological activities, especially for skeletal myogenesis [1]. It is an embryonic regulator of myogenesis and an autocrine factor that promotes myoblast differentiation in vitro [2-4]. Knockdown of IGF2 in mouse muscle myoblasts impaired the differentiation progress, demonstrating that IGF2 regulates muscle stem cell differentiation [3]. Its dysregulation has been linked to multiple disorders, including Silver-Russell syndrome and Beckwith-Wiedemann syndrome. Defective IGF2 signaling pathway or overexpression of IGF2 can trigger uncontrolled cell growth and lead to cancer growth. IGF2, suggested as a potential biomarker for various stages of tumor progression, plays a critical role in cancer development due to its abnormal expression in tumor tissues. However, despite extensive study into the expression of IGF2 and its induced signal transduction pathway, the mechanisms governing the secretion of newly synthesized IGF2 from producing cells are poorly understood.

IGF2 operates by binding to specific cell surface receptors, thereby initiating downstream signaling pathways, To inhibit IGF2 signaling, strategies can focus on targeting receptors integral to this process, such as the IGF1 receptor (IGF1R), which plays a crucial role in transmitting IGF2 signals. Preclinical therapeutic approaches using IGF1R inhibitors have demonstrated anti-tumor effects. Alternatively, the IGF2 signaling can be limited by blocking the secretion of newly synthesized IGF2 proteins. Therefore, there is a need for effective approaches that target the IGF2 signaling pathway.

### BRIEF SUMMARY OF THE INVENTION

In one aspect, the subject invention provides a pharmaceutical composition for inhibiting IGF2 signaling comprising an inhibitor of the IGF2-TMED10 interaction in a cell of a subject having abnormal IGF2 signaling.

In another aspect, the subject invention provides a method of treating a subject having abnormal IGF2 signaling, where the method comprises: (a) providing a pharmaceutical composition comprising an inhibitor of the IGF2-TMED10 interaction in a cell; and (b) administering an effective amount of the composition to the subject, and optionally one or more pharmaceutical carriers and/or excipients.

In yet another aspect, the subject invention provides a method of treating cancer in a subject in need thereof, the method comprising: (a) providing a pharmaceutical composition comprising an inhibitor of the IGF2-TMED10 interaction in a cell; and (b) administering an effective amount of the composition to the subject, and optionally one or more pharmaceutical carrier or an excipient.

In preferred embodiments, the inhibitor comprises a human IGF2 polypeptide comprising residues 112-140 of IGF2 sequence, a human TMED10 polypeptide that comprises residues 1-130 of the human TMED10 sequence, a small molecule, a covalent inhibitor, or an antibody, and combinations thereof.

In certain embodiments, the subject is affected by Beckwith-Wiedemann syndrome, Silver-Russell syndrome, or Doege-Potter syndrome. In certain embodiments, the subject is affected by a cancer. In certain embodiments, the subject is a mammal, the mammal can be a human.

In certain embodiments, a genome editing tool may be used to inhibit the interaction between IGF2 and TMED10 in a cell. In certain embodiments, the genome editing tool includes, but is not limited to, a CRISPR/CAS9 (RNA-guided targeted) genome editing tool, where the CRISPR/CAS9 editing tool is used to mutate the IGF2 sequence that codifies for residues 112-140 of IGF2 or the human TMED 10 sequence that codifies for residues 1-130 of the human TMED10 sequence, wherein the mutated TMED10 blocks the TMED10 binding to IGF2.

In preferred embodiments, the secretion of IGF2 in the cell is blocked by the inhibitor.

### BRIEF DESCRIPTION OF THE DRAWINGS

FIGS. 1A-1I illustrate that knockdown of TMED10 causes defects in secretion of IGF2. FIG. 1A shows a diagram demonstrating the design of the RUSH-IGF2-HA construct and the RUSH assay. FIG. 1B show HeLa cells transfected with plasmids encoding Str-KDEL and full-length RUSH-IGF2-HA. Day 1 after transfection, cells were pre-incubated with cycloheximide for 2 hr. Then, the localization of RUSH-IGF2-HA was analyzed after incubating with biotin and cycloheximide for the indicated time. The view of the indicated area in panel E at a higher exposure was shown in panel E′. FIG. 1C shows HeLa cells transfected with control siRNA or siRNA against TMED10. Day 2 after transfection, the level of TMED10 and R actin in cell lysates were analyzed by immunoblot. FIG. 1D shows 24 hr after transfection with siRNAs, cells re-transfected with plasmids encoding Str-KDEL and RUSH-IGF2-HA. On day 3 after knockdown, cells were pre-incubated with cycloheximide for 2 hr. Then, cells were incubated with biotin and cycloheximide for 2 hr. After biotin incubation, the level of RUSH-IGF2-HA in the medium and in cell lysates were analyzed by immunoblot. FIG. 1E shows quantification of the abundance of secreted IGF2 normalized to the abundance detected in the cell lysate group in the absence of biotin (mean±S.D.; n=3). FIG. 1F shows 24 hr after transfection with siRNAs, cells re-transfected with plasmids encoding Str-KDEL and RUSH-ShhN-HA. On day 3 after knockdown, cells were pre-incubated with cycloheximide for 2 hr. Then, cells were incubated with biotin and cycloheximide for 2 hr. After biotin incubation, the level of RUSH-ShhN-HA in the medium and in cell lysates were analyzed by immunoblot. FIG. 1G shows quantification of the abundance of secreted ShhN normalized to the abundance detected in the cell lysate group in the absence of biotin (mean±S.D.; n=3). FIG. 1H shows day 1 after transfection with siRNAs, cells transiently transfected RUSH-HA-IGF2. On day 3 after knockdown, cells were pre-incubated with cycloheximide for 2 hr. Then, cells were incubated with biotin and cycloheximide for 2 hr. After biotin incubation, the abundance of RUSH-HA-IGF2 in the medium and in cell lysates were analyzed by immunoblot. FIG. 1I shows quantification of the abundance of secreted IGF2 normalized to the abundance detected in the cell lysate in the absence of biotin (mean±S.D.; n=3). In each experimental group of each replicated experiment, the value was normalized to the average value of the Mock group and the TMED10 KD group (H, J, L). ***, p<0.001; ****, p<0.0001; n.s., not significant.

FIGS. 2A-1I illustrate that TMED10 mediates the release of IGF2 into COPII vesicles. FIG. 2A shows HeLa cells transfected with control siRNA or siRNA against TMED10. Day 1 after transfection, cells were re-transfected with plasmids encoding Str-KDEL and RUSH-IGF2-HA. On day 3 after knockdown, cells were pre-incubated with cycloheximide for 2 hr. Then, cells were incubated with biotin and cycloheximide for 20 min and the localization of IGF2 was analyzed. Size bar, 10 μm. FIG. 2B shows, concurrently, the abundance of TMED10 and R actin in cell lysates before biotin treatment analyzed by immunoblot. A representative example of three biological repeats was shown in this panel. FIG. 2C shows quantification of the percentage of cells showing juxtanuclear-localized RUSH-IGF2-HA at the indicated time point after biotin treatment (mean±S.D.; n=3; >100 cells counted in each experiment). FIG. 2D shows a diagram demonstrating the vesicle formation assay. FIGS. 2E, 2F, and 2H illustrate the vesicle formation assay performed using HEK293T cells (FIG. 2E) or HEK293T cells transfected with control siRNA or siRNA against TMED10 (FIGS. 2F and 2H). Vesicle fractions were analyzed by immunoblot. FIGS. 2G and 2I shows quantification of the budding efficiency of the indicated proteins from the vesicle formation assay (mean±S.D.; n=3). The budding efficiency was quantified by calculating the abundance of RUSH-IGF2-HA in the vesicle fraction normalized to the level of the cargo protein in 1% loading. The value was then normalized to the average value of all experimental groups in each replicated experiment. **, p<0.01; ***, p<0.001.

FIGS. 3A-3D show that the residues 112-140 in IGF2 are required for ER-to-Golgi trafficking of IGF2. FIG. 3A shows sequence alignment of IGF2 from different species (SEQ ID NOs: 6-11). FIG. 3B shows HeLa cells transfected with plasmids encoding the indicated RUSH constructs incubated with biotin and cycloheximide for the indicated period of time. The localizations of the indicated proteins were analyzed by immunofluorescence. Size Bar, 10 μm. FIGS. 3C and 3D illustrate the quantified percentage of cells showing juxtanuclear localization patterns of the RUSH construct after biotin treatment (mean±S.D.; n=3; >100 cells counted in each experiment). **, p<0.01; ***, p<0.001; ****, p<0.0001; n.s., not significant.

FIGS. 4A-4H show that the residues 112-140 in IGF2 directly interact with the GOLD domain of TMED10. FIGS. 4A, 4C and 4E show HEK293T cells co-transfected with plasmids encoding the indicated constructs. Day 1 after transfection, the cells were treated with 2 mM DSP, and the cell lysates were incubated with beads conjugated with anti-FLAG antibodies. The bound proteins were analyzed by immunoblot. FIGS. 4B, 4D, and 4F show quantification of relative levels of indicated proteins that co-immunoprecipitated with the FLAG-tagged proteins (mean±S.D.; n=3). The quantification was performed by calculating the abundance of the bound protein normalized to the abundance of the protein in the loading. The value was then normalized to the average value of all of the experimental groups in each biological repeat. FIG. 4G shows peptides corresponding to the 112-140 residues in IGF2 covalently linked to thiopyridone sepharose 6B, incubated with purified GST or GST-tagged human TMED10 GOLD domain (residues 1-130). After incubation, the bound proteins were analyzed by immunoblot. FIG. 4H shows levels of GST-TMED10¹⁻¹³⁰ bound to the IGF2 peptides quantified (mean±S.D.; n=3). The quantification is normalized to the average level of GST and GST-TMED10 (1-130) that bound to the IGF2 peptides in each biological repeat. *, p<0.05; **, p<0.01; ****, p<0.0001.

FIGS. 5A-5D show that TMED10 plays important roles in the secretion of IGF2 in C2C12 cells for muscle stem cell differentiation. FIG. 5A shows C2C12 cells transfected with control siRNA or siRNA against TMED10 incubated with the differentiation medium (DM) with or without 100 ng/mL purified IGF2. After incubation for 3 days, the expression of the indicated proteins was analyzed by immunoblot. FIG. 5B shows relative levels of the indicated proteins after cell differentiation assay were quantified (mean±S.D.; n=3). The quantification is normalized to the average level of myogenin in all of the three experimental groups in each biological repeat. FIG. 5C shows C2C12 cells transfected with control siRNA or siRNA against TMED10 incubated with the DM with or without 100 ng/mL purified IGF2 for 3 days. The morphology of myotube that labeled with MHC was analyzed by immunofluorescence. Size Bar, 200 μm. FIG. 5D shows quantification of the differentiation index in each of the indicated experimental group (mean±S.D.; n=3). The differentiation index was quantified by calculating the percentage of the number of nuclei in myosin heavy chain (MHC)-positive cells versus the total number of nuclei after the C2C12 differentiation assay. ***, p<0.001; **, p<0.01.

FIGS. 6A-6G illustrate that TMED10 regulates ER-to-Golgi transport of sortilin. FIG. 6A is a table showing the list of transmembrane proteins that are less represented in the vesicle fraction in the TMED10 KO group compared to the WT group (average fold change <0.5). FIG. 6B shows the vesicle formation assay performed using HeLa WT or HeLa TMED10 KO cells. The indicated proteins were analyzed by immunoblot. FIG. 6C shows WT or TMED10 KO HeLa cells transfected with SBP-EGFP-sortilin and Str-KDEL in the presence or absence of TMED10-FLAG. 24 h after transfection, cells were pre-incubated with cycloheximide for 2 hr. Then, cells were incubated with biotin and cycloheximide for the indicated time points. The localizations of the indicated proteins were then analyzed by immunofluorescence. Size bar, 10 μm. FIG. 6D shows quantification of the percentage of cells showing juxta-nuclear-located sortilin (mean±S.D.; n=3; >100 cells counted for each experiment). ***p<0.001; ****p<0.0001; n.s., not significant. FIG. 6E shows a diagram demonstrating the vesicle immunoprecipitation assay. FIGS. 6F and 6G show the vesicle formation assay performed in untransfected HeLa cells or in cells co-transfected with plasmids encoding TMED10-HA and RUSH-sortilin-Myc. Subsequently, vesicles enriched with TMED10-HA were immunoisolated and the abundance of the indicated proteins in the immunoisolated vesicles and the abundance of the indicated proteins in the vesicles that were not immunoisolated (flow through) were analyzed by immunoblot. Data shown in FIGS. 6F and 6G are representative example of three biological repeats.

FIGS. 7A-7G illustrate that sortilin regulates TGN export of IGF2. FIG. 7A shows HeLa cells transfected with control siRNA or siRNA against sortilin. Day 2 after transfection, the level of the indicated proteins in cell lysates were analyzed by immunoblot. FIG. 7B shows HeLa cells transfected with control siRNA or siRNA against sortilin. 24 hr after transfection, cells were re-transfected with plasmids encoding Str-KDEL and RUSH-HA-IGF2. On day 3 after knockdown, cells were pre-incubated with cycloheximide for 2 hr. Then, cells were incubated with biotin and cycloheximide for 2 hr. After biotin incubation, the level of RUSH-HA-IGF2 in the medium and in cell lysates were analyzed by immunoblot. FIG. 7C shows quantification of the abundance of secreted IGF2 normalized to the abundance detected in the cell lysate group (mean±S.D.; n=3). The value in each experimental group was normalized to the average value in the Mock group and the sortilin KD group in each biological repeat. FIG. 7D shows HeLa cells transfected with control siRNA or siRNA against sortilin. 24 hr after transfection, cells were re-transfected with plasmids encoding Str-KDEL and RUSH-HA-IGF2. On day 3 after knockdown, cells were pre-incubated with cycloheximide for 2 hr. Then, cells were incubated with cycloheximide and biotin for the indicated time and the localization of RUSH-HA-IGF2 was analyzed. Size bar, 10 μm. The magnified view of the indicated area in panels F and I are shown in panels F′, F″, I′, I″. FIG. 7E shows quantification of the percentage of cells showing juxtanuclear-located RUSH-HA-IGF2 (mean±S.D.; n=3; >100 cells counted in each experimental group). FIG. 7F shows quantification of the percentage of cells showing punctate patterns of RUSH-HA-IGF2 (mean±S.D.; n=3; >100 cells counted in each experimental group). FIG. 7G shows the proposed model demonstrating the dual functions of TMED10 in mediating IGF2 trafficking along the secretory pathway: (1) the GOLD domain of TMED10 recognizes the 112-140 residues of IGF2 to enrich IGF2 into COPII vesicles; (2) TMED10 also regulates ER export of newly synthesized sortilin, which is important for TGN-to-plasma membrane trafficking of IGF2. ***, p<0.001; n.s., not significant.

FIGS. 8A-8D illustrate knockout of TMED10 causing defects in secretion of IGF2. FIG. 8A shows cell lysates of WT or TMED10 KO HeLa cells analyzed by immunoblotting with the indicated antibodies. FIG. 8B shows WT or TMED10 KO HeLa cells transiently transfected with RUSH-IGF2-HA. Day 1 after transfection, cells were pre-incubated with cycloheximide for 2 hr. Then, cells were incubated with biotin and cycloheximide for 2 hr. After biotin incubation, the level of RUSH-IGF2-HA in the medium and in cell lysates were analyzed by immunoblot. FIG. 8C shows quantification of the abundance of secreted IGF2 normalized to the abundance detected in the cell lysate group (mean±S.D.; n=3). In each replicated experiment, the value in each experimental group was normalized to the average value of the WT and TMED10 KO group. **, p<0.01. FIG. 8D shows TMED10 KO HeLa cells transiently transfected with RUSH-HA-IGF2. Day 1 after transfection, cells were pre-incubated with cycloheximide with/without 100 nM Bafilomycin A1 or 50 μM MG132 for 2 hr. Cells were incubated with biotin for 2 hr. After incubation, the level of RUSH-IGF2-HA in cell lysates were analyzed by immunoblot.

FIGS. 9A-9D illustrate that knockout of TMED10 causes defects in ER-to-Golgi trafficking of IGF2. FIGS. 9A and 9C show WT or TMED10 KO HeLa cells transiently transfected with plasmids encoding the RUSH-IGF2-HA (A-R and T-Y) or co-transfected with plasmids encoding RUSH-IGF2-HA and TMED10-FLAG (Z-AB). Day 1 after transfection, cells were pre-incubated with cycloheximide for 2 hr. Then, cells were incubated with biotin and cycloheximide at 37° C. for indicated time points and the localization of RUSH-IGF2-HA was analyzed by immunofluorescence. Size Bar, 10 μm. FIGS. 9B and 9D illustrate the percentage of cells showing juxta-nuclear localization patterns of RUSH-IGF2-HA in each experimental group was quantified (mean±S.D.; n=3: >100 cells counted in each experiment). **, p<0.01; ***, p<0.001; n.s., not significant.

FIGS. 10A-10D illustrate TMED10-FLAG trafficking together with RUSH-IGF2-HA from the ER to the Golgi. FIGS. 10A and 10C show HeLa cells co-transfected with RUSH-IGF2-HA and TMED10-FLAG or co-transfected with RUSH-ShhN-HA and TMED10-FLAG. Day 1 after transfection, the localizations of the indicated proteins were analyzed after biotin treatment for 0 min (A-C, H-J) or 10 min (D-F, K-M). Size Bar, 10 μm. FIGS. 10B and 10D show quantification of the percentage of cells showing juxta-nuclear localization patterns of TMED10-FLAG in cells co-expressing TMED10-FLAG and RUSH-IGF2-HA or RUSH-ShhN-HA after biotin treatment for the indicated time (mean±S.D.; n=3; >100 cells counted in each experiment). ****, p<0.0001; n.s. not significant.

FIGS. 11A-11C illustrate the analysis of the colocalization between RUSH-IGF2-HA and TMED10-FLAG using a permeabilized cell assay. FIG. 11A shows HeLa cells co-transfected with RUSH-IGF2-HA and TMED10-FLAG. Day 1 after transfection, cells were permeabilized by digitonin and then incubated with rat liver cytosol, biotin and GTPγS at 37° C. for 15 min. After incubation, the localizations of the indicated proteins were analyzed by immunofluorescence. Size Bar, 10 μm. FIGS. 11B and 11C show magnified views of the indicated areas in panel C of FIG. 11A.

FIGS. 12A and 12B illustrate that residues 112-140 in IGF2 are sufficient to promote ER-to-Golgi trafficking. FIG. 12A shows HeLa cells transfected with plasmids encoding the indicated RUSH constructs incubated with biotin for the indicated period of time. The localizations of the indicated proteins were analyzed by immunofluorescence. Size Bar, 10 μm.

FIG. 12B shows quantification of the percentage of cells showing juxtanuclear localization patterns of the RUSH construct 20 min after biotin treatment (mean±S.D.; n=3; >100 cells counted in each experiment).

### BRIEF DESCRIPTION OF THE SEQUENCES

SEQ ID NO: 1
5′-GTGAGGAGATTCACAAGGA-3′ (target sequence
against human TMED10).
SEQ ID NO: 2
5′-GTCCTGTACTTCAGCATCT-3′ (target sequence
against mouse TMED10).
SEQ ID NO: 3
5′-GCACAATCTTTACCTCAGA-3′ (target sequence
against sortilin).
SEQ ID NO: 4
5′-TAACGGAAAAGGGCCGCGCC-3′ (sgRNA targeting
exon 1 of TMED10).
SEQ ID NO: 5
KFFQYDTWKQSTQRLRRGLPALLRARRGHC (Synthetic IGF2
112-140 peptide).
SEQ ID NO: 6
MGIPMGKSMLVLLTFLAFASCCIAAYRPSETLCGGELVDTLQFVCGDRG
FYFSRPASRVSRRSRGIVEECCFRSCDLALLETYCATPAKSERDVSTPP
TVLPDNFPRYPVGKFFQYDTWKQSTQRLRRGLPALLRARRGHVLAKELE
AFREAKRHRPLIALPTQDPAHGGAPPEMASNRK
SEQ ID NO: 7
MGIPVGKSMLVLLISLAFALCCIAAYGPGETLCGGELVDTLQFVCSDRG
FYFSRPSSRANRRSRGIVEECCFRSCDLALLETYCATPAKSERDVSTSQ
AVLPDDFPRYPVGKFFQYDTWRQSAGRLRRGLPALLRARRGRMLAKELK
EFREAKRHRPLIVLPPKDPAHGGASSEMSSNHQ
SEQ ID NO: 8
MGITAGKSMLALLAFLAFASCCYAAYRPSETLCGGELVDTLQFVCGDRG
FYFSRPSSRINRRSRGIVEECCFRSCDLALLETYCAAPAKSERDVSAST
TVLPDDFTAYPVGKFFQSDTWKQSTQRLRRGLPAFLRARRGRTLAKELE
ALREAKSHRPLIALPTQDPATHGGASSEASSD
SEQ ID NO: 9
MCAARQILLLLLAFLAYALDSAAAYGTAETLCGGELVDTLQFVCGDRGF
YFSRPVGRNNRRINRGIVEECCFRSCDLALLETYCAKSVKSERDLSATS
LAGLPALNKESFQKPSHAKYSKYNVWQKKSSQRLQREVPGILRARRYWQ
AEGLQAAEEARAMHRPLISLPSQRPPAPRASPEATGPQE
SEQ ID NO: 10
MEQLSCKHRSSSVEAEAQLCRQTESRSTQLPRMSVMRHLFLLSITFLVY
TLDSAKAYRATETLCGGELVDTLQFVCGDRGFYFSTNNGRSNRRPNRGI
VDVCCFKSCDLELLETYCAKPTKNERDVSTAPATAIPPLSKQDLYHKHH
HTKSSKYDIWQRKSIHRLRRGVPAIVRARQYRLLMEKAEEAEQALSHRP
LTTLPITRPLRLQQASEPSHN
SEQ ID NO: 11
MDDYHVFCASCRKTEETRTTMRSLIVFVLSLSMLISNVTAGETLCGGEL
VDTLQFVCGEDGFYISRPNRSNSRRPQRGIVEECCFRSCELHLLQQYCA
KPVKSERDVSSTSLQVFPVSQALHKDTINVKYSKYEVWQQKAAQRLRRG
VPSILLARKFRRQMEKIQDEEQTSFHRPLMTLPNRQPAIVPHVQISTSR
K

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts, the term “about” is used provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic compound. As used herein, the term “animal” may be, for example, humans, pigs, horses, goats, cats, dogs, apes, chimpanzees, orangutans, guinea pigs, hamsters, cows, or sheep. These benefits can include, but are not limited to, the treatment of a health condition, disease, or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the term “treatment” refers to eradicating, reducing, ameliorating, or reversing a sign or symptom of a health condition, disease, or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, the terms “therapeutically-effective amount,” “therapeutically-effective dose,” “effective amount,” and “effective dose” are used to refer to an amount or dose of a compound or composition thereof that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the terms “arresting”, “reducing”, “inhibiting”, “blocking”, “preventing”, “alleviating”, or “relieving” when referring to a compound, mean that the compound brings down the occurrence, severity, size, volume or associated symptoms of dental caries and/or carious lesions by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, or 100% compared to how cancer or cancer lesions would normally exist without application of the compound or a composition comprising the compound.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

The term “pharmaceutically acceptable” as used herein means compatible with the other ingredients of a pharmaceutical composition and not deleterious to the recipient thereof.

As used herein, the terms “oligo”, “oligonucleotide” are used interchangeably to describe short single strands of synthetic DNA or RNA, such as, for example, about a 5 nucleic acid base sequence to about a 500 nucleic acid base sequence.

As used herein, “vector” refers to a DNA molecule such as a plasmid for introducing a nucleotide construct, for example, a DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide a selectable characteristic, such as tetracycline resistance, hygromycin resistance or ampicillin resistance.

In this application, the terms “peptide”, “polypeptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “label” and like terms refer to a composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA), biotin, enzymes acting on a substrate (e.g., horseradish peroxidase), digoxigenin, ³²P and other isotopes, haptens, and proteins which can be made detectable, e.g., by incorporating a fluorescent label into the peptide or used to detect antibodies specifically reactive with the peptide. The term includes combinations of single labeling agents, e.g., a combination of fluorophores that provides a unique detectable signature, e.g., at a particular wavelength or combination of wavelengths. In the context of detecting nucleic acids (e.g., target sequences), the probes can, typically, be labeled with radioisotopes, fluorescent labels (fluorophores), or luminescent agents.

Any compositions or methods provided herein can be combined with one or more of any of the other compositions and methods provided herein.

Other features and advantages of the invention will be apparent from the following description of the preferred embodiments thereof, and from the claims.

All references cited herein are hereby incorporated by reference in their entirety.

The subject invention relates to novel compositions and methods for blocking the interaction between TMED10 and IGF2, either by mutating the 112-140 residues on IGF2 or mutating the GOLD motif on TMED 10, or utilizing a polypeptide that contains the GOLD domain of human TMED10, or small chemical molecules that block the interaction between TMED10 and IGF2, as an effective way to inhibit secretion of IGF2, thereby inhibiting downstream signaling, TMED10, a p24 family cargo receptor, regulates IGF2 secretion by recognizing an export motif on IGF2. Detailed mechanistic analysis revealed direct interaction between the GOLD domain of TMED10 and residues 112-140 of IGF2.

In certain embodiments, blocking the interaction between TMED10 and IGF2 provides a novel therapeutic strategy to inhibit IGF2 signaling to halt the progression of cancer or neurodevelopmental disorders associated with IGF2 dysregulation. In fact, the binding motifs of TMED10 and IGF2 are critical to the interaction between IGF2 and TMED10. In certain embodiments, mutations of these critical motifs can be used as novel ways to manipulate IGF2 secretion for cancer treatment. In addition, a single-pass transmembrane protein from the vacuolar protein sorting 10 protein family, sortilin, mediates the post-Golgi trafficking of IGF2.

The existing antagonist of IGF2 focuses on blocking the interaction between IGF2 and its receptor such as IGF1R. While IGF2 antagonists showed potential therapeutic significance, it's challenging to achieve sufficient and consistent efficacy in inhibiting IGF2 signaling. The specificity is also an issue due to the similarity between IGF2 and IGF1 or the IL family proteins. Rather, effective antagonists should selectively bind to IGF2 without interfering with the function of other related growth factors or receptors, for example the insulin receptor. Yet, there's no antagonist that focuses specifically on the intracellular trafficking of IGF2.

The dysregulation of IGF2 signaling pathway has been linked to multiple disorders. IGF2 is known to play a role in muscle growth and development, and it has been studied in the context of muscle regeneration and repair. Blocking the secretion of IGF2 leads to defective muscle stem cell differentiation. TMED10 mediates secretion of IGF2 and further regulates C2C12 differentiation in an autocrine manner. In certain embodiments, blocking IGF2 secretion by inhibiting the interaction between TMED10 and IGF2 is an effective way to downregulate IGF2 signaling and block cancer progression.

The secretion of IGF2 depends on the direct interaction between residues 112-140 of IGF2 and TMED10. TMED10 functions as a cargo receptor to mediate ER export of IGF2 for myoblast differentiation. The GOLD domain of TMED10 is also critical to IGF2 secretion. Moreover, TMED10 regulates ER export of sortilin, and sortilin is important for TGN export of IGF2.

After being synthesized from ribosomes, IGF2 needs to be delivered along the secretory transport pathway to perform its physiological functions. IGF2 is first synthesized as a precursor hormone containing 180 amino acids. After being imported into the ER, an N-terminal signal peptide is cleaved, generating pro-IGF2 (IGF2₂₅₋₁₈₀). The correctly folded pro-IGF2 proteins are then packaged into transport vesicles to be delivered to the Golgi apparatus. At the Golgi, pro-IGF2 undergoes O-glycosylation modifications and endoproteolysis, generating IGF2 peptides IGF2₂₅₋₁₂₈, IGF2₂₅₋₁₁₁ and the mature IGF2 (IGF22-91).

Coat protein complex II (COPII) is the key player that regulates the packaging of cargo proteins into vesicles at the ER. In the conventional secretory transport pathway, soluble cargo proteins in the lumen of the ER cannot be directly recognized by the COPII coat; instead, these cytosolic proteins are thought to be transported under the recognition of transmembrane cargo receptors. ERGIC53 is a major cargo receptor that recruits a variety of soluble cargo proteins to COPII vesicles in mammals. In addition, the p24 proteins play crucial roles in ER-Golgi bidirectional transport.

In certain embodiments, provided herein is a pharmaceutical composition for inhibiting IGF2 secretion by inhibiting the interaction between IGF2 and TMED10 in a cell of a subject that presents abnormal IGF2 signaling.

In other embodiments, provided herein is a method of treating a subject having abnormal IGF2 signaling by providing a pharmaceutical composition comprising an inhibitor of the IGF2-TMED10 interaction in a cell and administering an effective amount of the composition to the subject, and one or more pharmaceutical carrier or an excipient. In preferred embodiments, the inhibitor comprises a human IGF2 polypeptide comprising residues 112-140 of IGF2 sequence or a human TMED10 polypeptide that comprises residues 1-130 of the human TMED10 sequence, and combinations thereof. In certain embodiments, the inhibitor comprises a human TMED10 polypeptide that comprises residues 32-132 of the GOLD domain of the human TMED10 sequence. In certain embodiments, the inhibitor includes, but is not limited to, a small molecule, a covalent inhibitor, or an antibody, and combinations thereof.

In further embodiments, provided herein is a method of treating cancer in a subject in need thereof by providing a pharmaceutical composition comprising an inhibitor of the IGF2-TMED10 interaction in a cell and administering an effective amount of the composition to the subject, and one or more pharmaceutical carriers, or excipients. In preferred embodiments, the inhibitor comprises a human IGF2 polypeptide comprising residues 112-140 of IGF2 sequence or a human TMED10 polypeptide that comprises residues 1-130 of the human TMED10 sequence, and combinations thereof. In certain embodiments, the inhibitor comprises residues 120-140 of the IGF2-TMED10 sequence. In further embodiments, the inhibitor comprises residues 125-140 of the IGF2-TMED10 sequence. In certain embodiments, the inhibitor includes, but is not limited to, a small molecule, a covalent inhibitor, or an antibody, and combinations thereof.

In preferred embodiments, the compositions and methods of the subject invention inhibit the secretion of IGF2 in a cell having abnormal IGF2 signaling.

In certain embodiments, the nucleotide sequence of SEQ ID Nos: 1, 2, 3, and 4 also comprise sequences that are 99%, 98%, 97%, 96%, 95%, 94%, 93%, 92%, 91%, 90%, 89%, 88%, 87%, 86%, 85%, 84%, 83%, 82%, 81%, 80%, 75%, and 70% homologous. In certain embodiments, the amino acid sequence of peptide SEQ ID No: 5 also comprises sequences that are 99%, 98%, 97%, 96%, 95%, 94%, 93%, 92%, 91%, 90%, 89%, 88%, 87%, 86%, 85%, 84%, 83%, 82%, 81%, 80%, 75%, and 70% homologous.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g., carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. In addition, a filler, an anti-coagulant, a lubricant, a wetting agent, a fragrance, an emulsifier, a preservative, and the like may further be included. The use of carriers and/or excipients in the field of drugs and supplements is well known. Except for any conventional media or agent that is incompatible with the target health-promoting substance or with the adjuvant composition, carrier or excipient use in the subject compositions may be contemplated.

In one embodiment, the pharmaceutical composition can be formulated for administration via injection, for example, as a solution or suspension. The route of administration of the pharmaceutical composition of the present invention may also include, but is not limited to, oral, intravenous, intramuscular, intraarterial, intramedullary, intradural, intracardiac, transdermal, subcutaneous, intraperitoneal, intranasal, intestinal, topical, sublingual, or rectal route. The term “parenteral” is meant to include subcutaneous, transdermal, intravenous, intramuscular, intra-articular, intra-synovial, intrasternal, intradural, intralesional, and intra-cranial injection or infusion techniques. The pharmaceutical composition of the present invention may also be formulated as suppositories for intrarectal administration.

The solution or suspension can comprise suitable non-toxic, parenterally acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

The pharmaceutical composition of the present invention may vary depending on a variety of factors, including the activity of a certain active ingredient used, the subject's age, body weight, general health status, sex, diet, time of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. Preferably, taking all of the above factors into consideration, it is possible to administer an amount that can obtain the maximum effect with a minimum amount without side effects, and more preferably, it may be administered repeatedly several times a day at an effective dose of 1 to 10000 μg/body weight kg/day, even more preferably 10 to 1000 mg/body weight kg/day. The above dosage does not limit the scope of the present invention in any way.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, 50, 60, 70, 80, 90, 100 or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, 2 months, 3 months, 6 months, 9 months, 1 year, 1.5 years, 2 years, 2.5 years, 5 years or more than 10 years. The frequency and duration of administration of multiple doses of the compositions is such as prevent or treat kidney disease or inflammatory reactions. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of testing for kidney disease, such as, for example, glomerular filtration rate. In some embodiments of the invention, the method comprises administration of the compounds at several times per day, including but not limiting to 2 times per day, 3 times per day, and 4 times per day.

In some embodiments of the invention, the method comprises administration of multiple doses of the compositions of the subject invention. The method may comprise administration of therapeutically effective doses of a composition comprising the compound or composition thereof of the subject invention as described herein once a day, once a week, once a month, once a quarter, twice a year, once a year, or a lower frequency. Moreover, treatment of a subject with a therapeutically effective amount of the compositions of the invention can include a single treatment or can include a series of treatments. It will also be appreciated that the effective dosage of a compound or composition thereof used for treatment may increase or decrease over the course of a particular treatment. Changes in dosage may result and become apparent from the results of diagnostic assays to determine the presence of cancer cells, which are known in the art.

In one embodiment, the cell is a cancerous or tumorous cell, which is selected from, for example, prostate cancer cells, gallbladder cancer cells, intrahepatic biliary tract cancer cells, biliary tract cancer cells, oral cancer cells, pharyngeal cancer cells, laryngeal cancer cells, tongue cancer cells, duodenal cancer cells, eye tumor cells, mediastinal cancer cells, sinus cancer cells, renal pelvic cancer cells, heart cancer cells, glioblastoma cells, neuroblastoma cells, liver cancer cells, bone cancer cells, pancreatic cancer cells, skin cancer cells, head and neck cancer cells, breast cancer cells, lung cancer cells, skin or intraocular malignant melanoma cells, kidney cancer cells, uterine cancer cells, ovarian cancer cells, colon cancer cells, rectal cancer cells, anal region cancer cells, colorectal cancer cells, stomach cancer cells, testicular cancer cells, fallopian tube cancer endometrial carcinoma cells, cervical carcinoma cells, vaginal carcinoma cells, vulvar cancer cells, esophageal cancer cells, small intestinal cancer cells, endocrine system cancer cells, thyroid cancer cells, parathyroid cancer cells, adrenal gland cancer cells, soft tissue sarcoma cells, urethral cancer cells, penile cancer cells, childhood cancer cells, lymphocytic lymphoma cells, bladder cancer cells, ureter cancer cells, renal pelvic carcinoma cells, central nervous system (CNS) cancer cells, primary CNS lymphoma cells, spinal cancer cells, brainstem glioma cells, pituitary adenoma cells, Kaposi's sarcoma cells, epidermal cancer cells, squamous cell carcinoma cells, follicular lymphoma cells, immune large-cell lymphoma cells, mantle cell lymphoma cells, mycosis fungoides cells, hepatoblastoma cells, retinoblastoma cells, peritoneal cancer cells, brain tumor cells, thymic cancer cells, and any combination of the above cancer cells.

In some embodiments of the invention, the method comprises activating the compound of the subject invention with light irradiation. The method may comprise irradiating the cancer cells treated with the composition of the subject invention 1, 2, 3, 4, 5, 6, 7, 8, 9, 10, 15, 20, 25, 30, 35, 40, 50, 60, 70, 80, 90, 100 or more times. In some embodiments, the cancer is irradiated over the course of 1 day, 2 days, 3 days, 4 days, 5 days, 6 days, 7 days, 10 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 52 weeks, about 1.5 years, about 2 years, about 2.5 years, about 5 years, or more than 10 years. Moreover, treatment of a subject with irradiation of the cancer can include a single treatment or can include a series of treatments. It will also be appreciated that the effective excitation frequency, intensity, and time used for treatment may increase or decrease over the course of a particular treatment. Changes in excitation frequency, intensity, and duration may result and become apparent from the results of testing for cancer remission well known in the art. In some embodiments of the invention, the method comprises irradiation of the compounds at several times per day, including but not limiting to 2 times per day, 3 times per day, and 4 times per day. In preferred embodiments, the irradiation of at least one dose of the composition is repeated every day for about 2 weeks to about 10 weeks.

In some embodiments, a cancer or tumor being targeted by the subject invention is in any of a variety of stages including newly diagnosed, relapsed, refractory, progressive disease, remission, and others. In some embodiments, the cancer or tumor being treated is a newly diagnosed cancer. In some embodiments, the cancer or tumor is a recurrent cancer (e.g., a recurrent gynecologic cancer such as recurrent epithelial ovarian cancer, recurrent fallopian tube cancer, recurrent primary peritoneal cancer, or recurrent endometrial cancer). In some embodiments, the present invention is applicable to treatment of metastatic cancers.

In certain embodiments, the therapeutically effective amount of the composition of the invention can be administered through intraperitoneal administration or by sustained release systems, such as semipermeable matrices of solid hydrophobic polymers containing the compounds of the invention. Administration may be also by way of other carriers or vehicles such as patches, micelles, liposomes, vesicles, implants (e.g., microimplants), synthetic polymers, microspheres, nanoparticles, and the like. In certain embodiments, the compositions may be administered using a nanoparticle to passage the composition through skin.

The composition may further contain formulation agents such as suspending, stabilizing and/or dispersing agents. In further embodiments, the active ingredients of the compositions according to the instant invention may be in powder form, obtained by aseptic isolation of sterile solid or by lyophilization from solution for constitution with a suitable vehicle, e.g., sterile, pyrogen-free water, before use.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

Regardless of the route of administration selected, the composition may be formulated into pharmaceutically acceptable dosage form by conventional methods known to those of skill in the art. The composition may be formulated for administration in any convenient way for use in human or veterinary medicine, by analogy with other pharmaceuticals.

The compositions can further comprise one or more pharmaceutically acceptable carriers, and/or excipients, and can be formulated into preparations, for example, semi-solid or liquid forms, such as solutions or injections.

The formulations may conveniently be presented in unit dosage form and may be prepared any methods well known in the art of pharmacy. The amount of compound which can be combined with a carrier material to produce a single dosage form will vary depending upon the subject being treated, the particular mode of administration. The amount of an active ingredient which can be combined with carrier material to produce a single dosage form will usually be the amount of the compound which produces a therapeutic effect. Usually, out of one hundred percent, this amount will range from about 1 wt % to about 99 wt % of active ingredient, preferably from about 5 wt % to about 70 wt %, most preferably from about 10 wt % to about 30 wt %.

The above-described contents of the present invention are equally applied to each other as long as they do not contradict each other, and it is also included in the scope of the present invention that those skilled in the art can implement with appropriate changes.

Hereinafter, the present invention will be described in detail through Examples, but the scope of the present invention is not limited only to the Examples below.

### Materials and Methods

### Constructs, Reagents, Cell Culture, Immunofluorescence, and Transfection

The cDNA encoding human IGF2, human TMED10, human sortilin, and the plasmids encoding Str-KDEL_SBP-EGFP-HA-IGF2 were synthesized from BGI (Beijing, China). The plasmids encoding 3×HA-tagged IGF2 (IGF2-HA), GST-tagged IGF2, 3×FLAG-tagged TMED10 (TMED10-FLAG), Str-KDEL_SBP-EGFP-sortilin, Str-KDEL_SBP-EGFP-sortilin-myc, Str-KDEL_SBP-EGFP-IGF2-HA and truncated versions of IGF2 were generated by standard molecular cloning procedures. The plasmids encoding mutated version of IGF2 were generated by QuikChange II site-directed mutagenesis using plasmids encoding IGF2-HA or Str-KDEL_SBP-EGFP-IGF2-HA as templates. The plasmids encoding siRNA-resistant TMED10-FLAG were generated by QuikChange II site-directed mutagenesis using plasmid encoding TMED10-FLAG as template.

siRNAs against TMED10 were purchased from Ribo-bio (Guangzhou, China). The target sequence against human TMED10 is 5′-GTGAGGAGATTCACAAGGA-3′ (SEQ ID NO: 1). The target sequence against mouse TMED10 is 5′-GTCCTGTACTTCAGCATCT-3′ (SEQ ID NO: 2). The target sequence against sortilin is 5′-GCACAATCTTTACCTCAGA-3′ (SEQ ID NO: 3). The commercial antibodies were rabbit anti-HA (Cell Signaling, catalogue number 3724), rabbit anti-TMED10 (Proteintech, catalogue number 15199-1-AP), mouse anti-MHC (DSHB, catalogue number MF 20) and mouse anti-Myogenin (Santa Cruz Biotechnology, catalogue number SC-12732); sheep anti-TGN46 (BIO-RAD, catalogue number AHP500G), mouse anti-GM130 (BD Bioscience, catalogue number 610823); mouse anti-FLAG (Sigma, catalogue number F3165); mouse anti-β actin antibody (Proteintech, catalogue number 60008-1-Ig); goat anti-GST (GE Healthcare, catalogue number 27-4577-01); mouse anti-TMED2 (Santa Cruz Biotechnology, catalogue number SC-376459); mouse anti-Myc (Cell signaling, catalogue number 2276); rabbit anti-sortilin (Proteintech, catalogue number 12369-1-AP). Rabbit anti-Sec22B antibodies and rabbit anti-ERGIC53 antibodies were kindly provided by Prof. Randy Schekman (University of California, Berkeley, CA, USA). Rabbit anti-TMED7 antibodies were kindly provided by Prof. Pingbo Huang (Hong Kong University of Science and Technology, Hong Kong, SAR).

HeLa cells and HEK293T cell lines were kindly provided by the University of California-Berkeley Cell Culture Facility and were confirmed by short tandem repeat profiling. All cell lines were tested negative for Mycoplasma contamination. HeLa and HEK293T cells were cultured in Dulbecco's Modified Eagle Medium (DMEM) containing 10% fetal bovine serum and 1% penicillin streptomycin mix (Invitrogen). C2C12 cells were cultured in Dulbecco's Modified Eagle Medium (DMEM) containing 20% fetal bovine serum and 1% penicillin streptomycin mix (Invitrogen). Transfection of siRNA or DNA constructs into HeLa cells, or HEK293T cells and immunofluorescence were performed as described previously (55). Images were acquired with a Zeiss Axio Observer Z1 microscope system (Carl Zeiss, Germany) equipped with an ORCA Flash 4.0 camera (Hamamatsu, Japan) or Leica SP8 Confocal Microscope (Leica, Germany).

For CRISPR experiments, sgRNA sequences were ligated into AAV9-U6-Dsred plasmids. Clonal cell lines were derived by diluting cell suspensions to a single cell per well and expanding individual wells. Genotyping of clonal cell lines was performed by Sanger sequencing of target site PCR amplicons of genomic DNA isolated by Puregene kit (Qiagen). sgRNA was as follows: TMED10, 5′-TAACGGAAAAGGGCCGCGCC-3′ (SEQ ID NO: 4) targeting exon 1 of TMED10.

### Retention Using Selective Hook (RUSH) Assay

The RUSH assays were performed as described (21). Briefly, HeLa cells were transfected with plasmids encoding Str-KDEL and SBP-EGFP-sortilin or SBP-EGFP-sortilin-myc or different version of the RUSH constructs of IGF2. Day 1 after transfection, cells were incubated in complete medium with 100 ng/l cycloheximide for 2 hr. Then, the cells were incubated in complete medium containing 40 μM biotin (Sigma-Aldrich) and 100 ng/l cycloheximide (Sigma-Aldrich) for the indicated time. Subsequently, cells were fixed by 4% PFA and mounted on glass slides by ProLong™ Gold Antifade Mountant with DAPI (Invitrogen) for microscope analysis.

To analyze the secretion of IGF2, HeLa cells transfected with plasmids encoding Str-KDEL and different version of the RUSH constructs of IGF2 were incubated in complete medium with 100 ng/l cycloheximide for 2 hr. Then, cells were treated with 100 ng/l cycloheximide and M biotin in medium without FBS for the indicated time. Then, the secreted proteins were precipitated by TCA precipitation. The cells were collected and lysed by HKT buffer (100 mM KCl, 20 mM Hepes, pH 7.2, 0.5% Triton X-100). The secreted proteins and cell lysates were analyzed by immunoblot.

### Immunoprecipitation, Protein Purification, and Binding Assay

Immunoprecipitation of FLAG-tagged TMED10 or TMED10¹⁻¹³⁰ was performed by treating HEK293T cells co-transfected with plasmids encoding the indicated proteins in PBS containing 2 mM dithiobis[succinimidylpropionate](DSP) and 2 mM CaCl₂ at room temperature for 30 min, and then quenched with 25 mM Tris-HCl, pH 7.5. The cells were then lysed in lysis buffer (50 mM Tris-HCl, 150 mM NaCl, 2 mM CaCl₂, and 0.1% TX-100, supplemented with proteinase inhibitors (Roche), pH7.5). Subsequently, 500 μl of 0.5 mg/ml cell lysates were incubated with 10 μl of compact anti-FLAG agarose affinity beads at 4° C. overnight. M2-FLAG affinity beads was pre-blocked with 1 hr incubation in blocking buffer (50 mM Tris-HCl, 500 mM NaCl, 2 mM CaCl₂, 5% BSA, pH 7.5) to reduce nonspecific binding. After incubation, the beads were washed 3 times with 1 ml of blocking buffer (5% BSA) and 2 times with 1 ml of blocking buffer (-BSA), and the bound material was analyzed by immunoblot.

Purification of GST-tagged TMED10¹⁻¹³⁰ was performed as described previously [5]. GST pull-down assays were carried out with 10 μl of compact GSH beads bearing around 5 μg of GST-tagged TMED10¹⁻¹³⁰. The beads were incubated with 200 μl of 0.5 mg/ml of cell lysates from HEK293T cells transfected with indicated plasmids in HKT buffer with mixing at 4° C. overnight. After incubation, the beads were washed three times with 500 μl of HKT buffer and twice with 500 μl of HK buffer, and the bound material was analyzed by immunoblot.

Peptide binding assay was performed as previously described. Synthetic IGF2 112-140 peptide (KFFQYDTWKQSTQRLRRGLPALLRARRGHC) (SEQ ID NO: 5) was purchased from GenScript and coupled to thiopyridone-Sepharose 6B beads (Sigma-Aldrich) via the added C-terminal cysteine residue. For binding experiments, GST or GST-tagged TMED10¹⁻¹³⁰ at around the same abundance (˜5 pmol) was pre-incubated at 4° C. for 30 min in a total volume of 450 μl binding buffer (20 mM Hepes, pH 7.2, 250 mM sorbitol, 70 mM KOAc, 1 mM Mg(OAc)₂, and 1 mg/ml bovine serum albumin). The coupled beads were blocked by a 40-min incubation at 4° C. in 5 mM β-mercaptoethanol, 50 mM NaOAc, 0.5 M NaCl, pH 4.5, followed by washing steps and a 2 h incubation at 4° C. in binding buffer. After incubation, 250 μl buffer containing around 5 μl beads conjugated with peptides was added to the reaction mixture at 4° C. for 80 min. The beads were washed five times by incubating with binding buffer containing 0.5M KOAc and 0.1% Triton without BSA for 1.5 min, followed by washing with binding buffer containing 0.1% Triton without BSA for 3 times. Then, the beads were analyzed by immunoblot.

### In Vitro Vesicle Formation Assay

In vitro vesicular release assays were performed as described previously [6, 7] Briefly, HeLa cells were un-transfected or transfected with control siRNA or siRNA against TMED10. Day 1 after transfection, cells were transfected with plasmids encoding RUSH-IGF2-HA. Day 2 after knockdown, HEK293T cells were permeabilized in 3 ml of ice-cold KOAc buffer (110 mM potassium acetate, 20 mM Hepes, pH 7.2, 2 mM magnesium acetate) containing 40 mg/ml digitonin on ice for 5 min. The semi-intact cells were then sedimented by centrifugation at 300 g for 3 min at 4° C. The cell pellets were washed twice with 1 ml of KOAc buffer and resuspended in 100 μl of KOAc buffer. The budding assay was performed by incubating semi-intact cells (around 0.02 OD/reaction) with 2 mg/ml of rat liver cytosol in a 100 μl reaction mixture containing 200 mM GTP and an ATP regeneration system (40 mM creatine phosphate, 0.2 mg/ml of creatine phosphokinase, and 1 mM ATP) in the presence or absence of 0.5 mg of SAR1A (H79G). After incubation at 32° C. for 1 h, the reaction mixture was centrifuged at 14,000 g to remove cell debris and large membranes. The medium-speed supernatant was then centrifuged at 100,000 g to sediment small vesicles. The pellet fraction was then resuspended in 100 μl of 35% OptiPrep and overlaid with 700 μl of 30% OptiPrep and 30 μl of KOAc buffer. The samples were centrifuged at 55,000 rpm in a TLS55 rotor in a Beckman ultracentrifuge at 4° C. for 2 hr. After centrifugation, the top fraction was analyzed by SDS-PAGE and immunoblot.

The vesicle formation assay was then performed in a large scale using one 15 cm dish of WT HeLa cells or TMED10 KO HeLa cells to provide donor membranes. The vesicle fraction was then analyzed by label-free quantitative mass spectrometry using the procedure as previously described (25).

### Vesicle Immunoprecipitation (Vesicle-IP) Assay

One 10-cm dish of HeLa cells with or without transfection was used to provide semi-intact cells for the vesicle formation assay in each experimental group of the vesicle-IP assay. The semi-intact cells were incubated with 2 mg/ml of rat liver cytosol in a 1.8 ml reaction mixture containing 200 mM GTP and an ATP regeneration system at 32° C. for 1 h. The reaction mixture was centrifuged at 16900 g centrifugation for 20 min. The medium-speed supernatant was then incubated with 30 μl Pierce anti-HA Magnetic beads at 4° C. overnight with rotation. The next day, the beads were collected with a magnetic stand. The supernatant was collected and centrifuged at 100K g in an S120AT3 (Hitachi) rotor at 4° C. for 30 min to sediment vesicles as “flow-through”. The beads were washed with ice-cold KOAc buffer containing 1 M, 0.5 M, and 0.11 M potassium acetate respectively, each for 3 times. The proteins in the flow-through fraction and in the on beads fraction were analyzed by immunoblot.

### Muscle Stem Cell Differentiation Assay

Undifferentiated C2C12 cells were cultured with DMEM containing 20% FBS and 1% penicillin-streptomycin mix. To induce differentiation, C2C12 cells transfected with control siRNA or siRNA against TMED10 were incubated with DMEM containing 2% horse serum and 1% penicillin-streptomycin in the presence or absence of 100 ng/ml purified IGF2 (R&D Systems, Catalog number: 792-MG) for 3 days. Then, the cells were analyzed by immunoblot or immunofluorescence.

### Sample Preparation for the Mass Spectrometry Analysis of the Secreted Proteins by C2C12 Cells.

Undifferentiated C2C12 cells grown in 15 cm dishes were cultured with DMEM containing 20% FBS and 1% penicillin-streptomycin mix until the cells reached 80-90% confluence. Then, C2C12 cells were transfected with siRNA against GFP or siRNA against mouse TMED10. Day1 after transfection, cells were washed five times with PBS and then cultured in DMEM containing 1% penicillin-streptomycin mix for 3 days. The culture medium was collected, and cell debris was removed by centrifugation. Subsequently, TCA precipitation was performed to precipitate the secreted proteins from the medium. The precipitated proteins were analyzed by SDS-PAGE and Coomassie Blue (Bio-Safe™ Coomassie-G250) staining. Subsequently, in-gel digestion and label-free mass spectrometry were performed as described [21].

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—TMED10 Mediates ER-to-Golgi Trafficking and the Secretion of IGF2

The insulin-like growth factor 2 (IGF2) plays critical roles in cell proliferation, migration, differentiation and survival. Despite its importance, the molecular mechanisms mediating the trafficking of IGF2 along the secretory pathway remain unclear. Newly synthesized IGF2 needs to be delivered along the secretory transport pathway to perform its physiological functions. IGF2 is first synthesized as a precursor hormone containing 180 amino acids. After being imported into the ER, an N-terminal signal peptide is cleaved, generating pro-IGF2 (IGF2²⁵⁻¹⁸⁰). The correctly folded pro-IGF2 proteins are then packaged into transport vesicles to be delivered to the Golgi apparatus. At the Golgi, pro-IGF2 undergoes O-glycosylation modifications and endoproteolysis, generating IGF2 peptides IGF2²⁵⁻¹²⁸, IGF2²⁵⁻¹¹¹, and the mature IGF2 (IGF2²⁵⁻⁹¹¹) [8]. Coat protein complex II (COPII) is the key player that regulates the packaging of cargo proteins into vesicles at the ER. In the conventional secretory transport pathway, soluble cargo proteins in the lumen of the ER cannot be directly recognized by the COPII coat; instead, these cytosolic proteins are thought to be transported under the recognition of transmembrane cargo receptors [9]. ERIGIC53 is a major cargo receptor that recruits a variety of soluble cargo proteins to COPII vesicles in mammals [9]. In addition, the p24 proteins play crucial roles in ER-Golgi bidirectional transport. Some p24 family members function as cargo receptors to regulate ER export of specific GPI-anchored proteins and autotaxin in mammalian cells [9-10] and to mediate secretion of Wnt proteins in Drosophila [11-12]. Soluble cargo proteins are also proposed to enter the nascent COPII vesicles by default, a process referred to as bulk flow [13].

The trans Golgi network (TGN) is another important station in the secretory transport pathway. At the TGN, various cargo adaptors and receptors have been shown to capture cargo molecules into nascent vesicles [14]. Sortilin is one of the Golgi-localized cargo receptors that is crucial for the sorting of numerous proteins in the anterograde and retrograde pathways and is broadly involved in multiple physiological activities, including lipid metabolism, neuronal development, immune system, myogenesis, and diabetes [15-19]. Particularly, the noncoding genetic variants at a locus near gene encoding sortilin were significantly associated with LDL cholesterol and coronary artery disease in humans [20-21]. Further analysis indicates that sortilin interacts with apolipoprotein B100 (ApoB100) and modulate the secretion of ApoB100-containing lipoproteins in hepatocytes, but whether sortilin upregulates or downregulates lipoprotein secretion remain controversial [22-23].

Secretion of newly synthesized soluble signaling proteins from the producing cells is tightly related to the downstream signaling pathway in targeted cells. Although a series of factors are identified to regulate the expression of IGF2 such as mTOR, PLD1 and miR-125b [4, 24-26], the cargo receptors that regulate the biosynthetic trafficking of newly synthesized IGF2 remain largely unknown.

We also reconstituted the release of IGF2 into COPII vesicles through an in vitro vesicle formation assay to quantify the efficiency of cargo packaging. Through these approaches, we found that a p24 family protein, TMED10, functions as a cargo receptor to mediate ER export of IGF2 for myoblast differentiation. Moreover, we found that TMED10 regulates ER export of sortilin, and sortilin is important for TGN export of IGF2. These studies shed light on molecular mechanisms regulating IGF2 secretion along the secretory pathway for muscle stem cell differentiation.

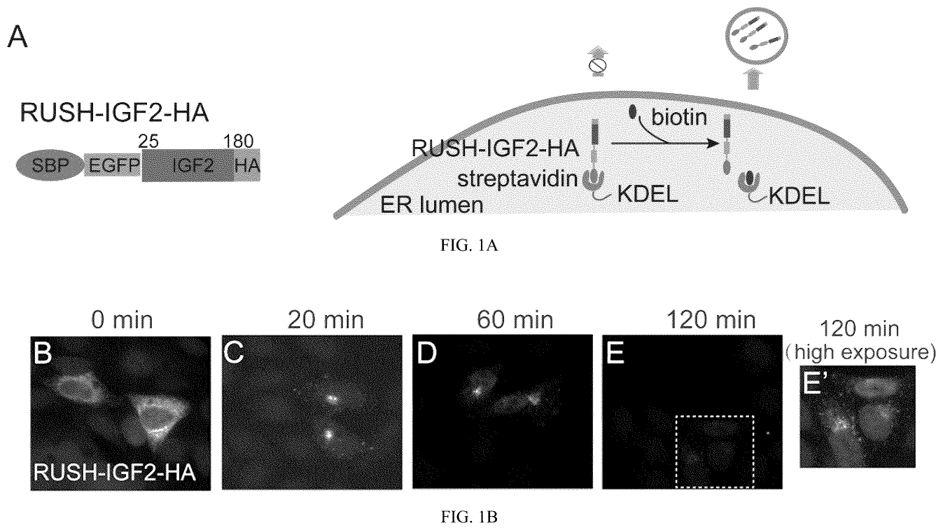

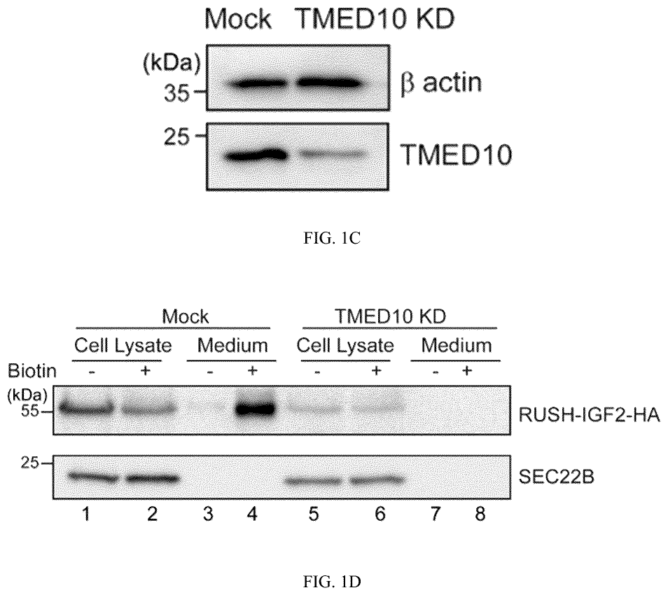

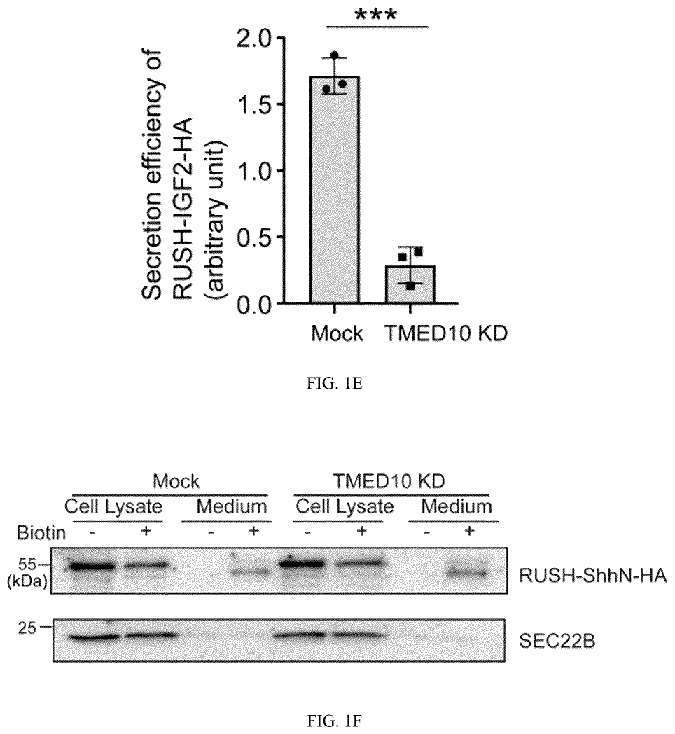

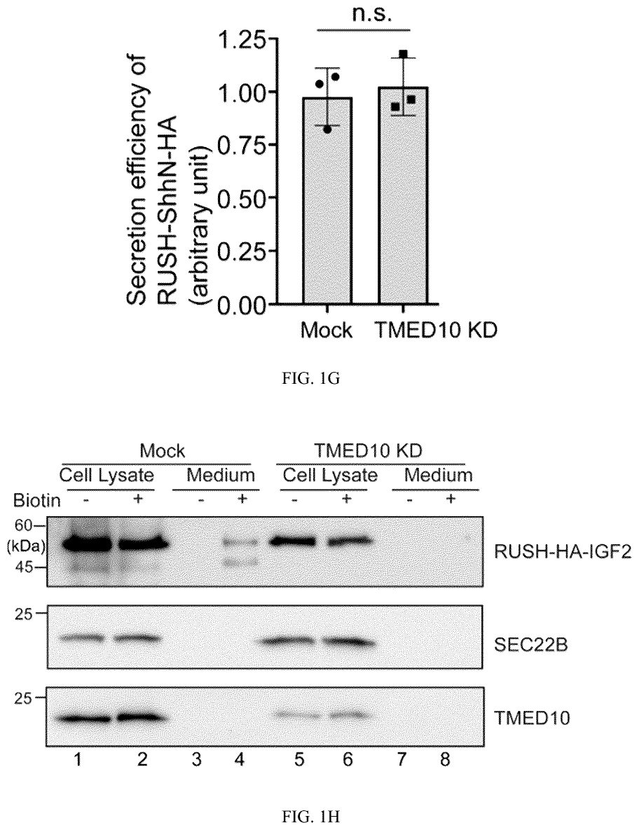

We performed co-immunoprecipitation (co-IP) experiments to reveal proteins that interact with HA-tagged N-terminal fragment of sonic hedgehog (ShhN-HA) or HA-tagged IGF2 (IGF2-HA) [27]. Label-free quantitative mass spectrometry analysis of the immunoprecipitated proteins identified a transmembrane protein, TMED10, which binds stronger to IGF2-HA than to ShhN-HA [27]. TMED10 is an ER- and Golgi-localized transmembrane protein that belongs to the p24 family. To test whether TMED10 is important for IGF2 secretion, we utilized a RUSH transport assay [27-29]. In this assay, HeLa cells were transfected with plasmids encoding human IGF2 (aa: 25-180, removed signal peptide) tagged with EGFP, the streptavidin binding peptide (SBP) and an HA tag at the C-terminus (referred to as RUSH-IGF2-HA or RUSH-IGF2²⁵⁻¹⁸⁰-HA) (FIG. 1A). This plasmid also encodes streptavidin fused to a C-terminal ER retention signal (Lys-Asp-Glu-Leu; Str-KDEL). Due to the binding between streptavidin and SBP, RUSH-IGF2-HA was retained at the ER upon expression (FIG. 1B, 0 min). It is noted that the RUSH system does not halt ER export but rather creates an imbalance, favoring retrieval over export. Consequently, most of the cargo remains at the ER in the absence of biotin. When cells were incubated with biotin, SBP was uncoupled with streptavidin, thereby releasing RUSH-IGF2-HA from the ER retrieval process (FIG. 1B in panels C-E′). Over 80% of cells showed Golgi-localized IGF2 when the cells were incubated with biotin for 20 min (FIG. 1B in panel C). After biotin treatment for 120 min, the signal of RUSH-IGF2-HA was greatly reduced (FIG. 1b in panels 1E-1E′), suggesting that IGF2 was secreted out of the cells or delivered to lysosomes for degradation.

We then performed an siRNA knockdown (KD) experiment to test whether TMED10 is required for IGF2 secretion. The expression of TMED10 was greatly reduced in HeLa cells transfected with siRNA against TMED10 (FIG. 1C). Remarkably, the secretion efficiency of RUSH-IGF2-HA was significantly decreased in TMED10 KD cells (FIG. 1D, compare lanes 4 and 8, and FIG. 1E), indicating that TMED10 plays an important role in IGF2 secretion. In contrast, the secretion of another protein, ShhN, was not affected in TMED10 KD cells (FIGS. 1F and 1G), demonstrating that TMED10 is a specific regulator for IGF2 rather than a common regulator.

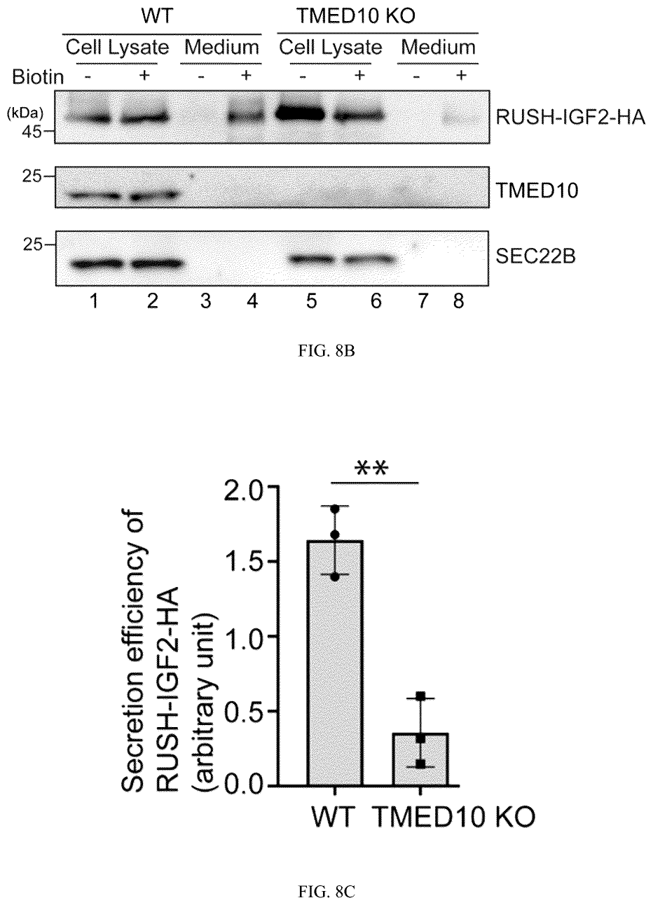

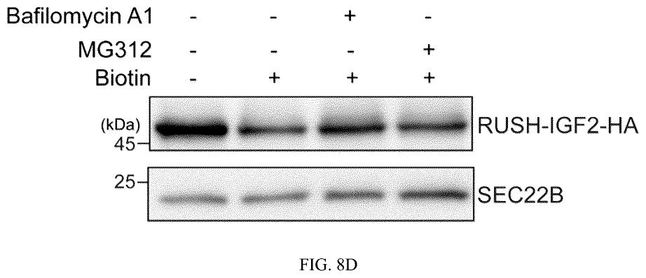

We then generated TMED10 knockout (KO) HeLa cells to test the effect of depleting TMED10 on IGF2 secretion. Western blot analysis indicates that TMED10 was completely depleted in TMED10 KO HeLa cells (FIG. 8A). The efficiency of RUSH-IGF2-HA secretion in TMED10 KO cells was also significantly reduced (FIG. 8B, compare lanes 4 and 8, and FIG. 8C), demonstrating that TMED10 is essential for IGF2 secretion.

We noticed that the size of the secreted RUSH-IGF2-HA detected by anti-HA antibodies is similar to the size of RUSH-IGF2-HA in cell lysates, suggesting the secreted RUSH-IGF2-HA we detected is not the cleaved form. A possible explanation is that we used antibodies detecting the HA tag at the C-terminus of IGF2 for the immunoblot analyses. We repeated this assay by using N-terminal HA-tagged RUSH-IGF2 (referred to as RUSH-HA-IGF2) to monitor the secretion of both big IGF2 and mature IGF2. We detected two bands in the medium group, and their molecular weights match the predicted molecular weights of the pro-IGF2 and mature IGF2 (FIG. 1H, lane 4). Consistent with the previous analyses, the secretion of both pro-IGF2 and mature IGF2 was reduced after TMED10 KD (FIG. 1H, compare lanes 4 and 8, and FIG. 1I).

We found that the abundance of RUSH-IGF2-HA in cell lysates from TMED10 KD or KO groups was decreased after biotin treatment (FIG. 1H, lanes 5-6, FIG. 8B, lanes 5-6). We hypothesize that some of the RUSH-IGF2-HA have been degraded rather than secreted. To test this, we performed the RUSH assay using TMED10 KO cells and treated the cells with the lysosomal inhibitor (bafilomycin A1), or the proteasome inhibitor (MG132) in the presence of biotin. We found that bafilomycin A1 treatment enhanced the abundance of RUSH-IGF2-HA in cell lysates after biotin treatment, whereas MG132 treatment only showed a modest effect (FIG. 8D), indicating the reduced RUSH-IGF2-HA protein levels in cell lysates after biotin treatment is mainly due to the lysosomal degradation.

### Example 2—TMED10 is Important for Packaging IGF2 into COPII Vesicles

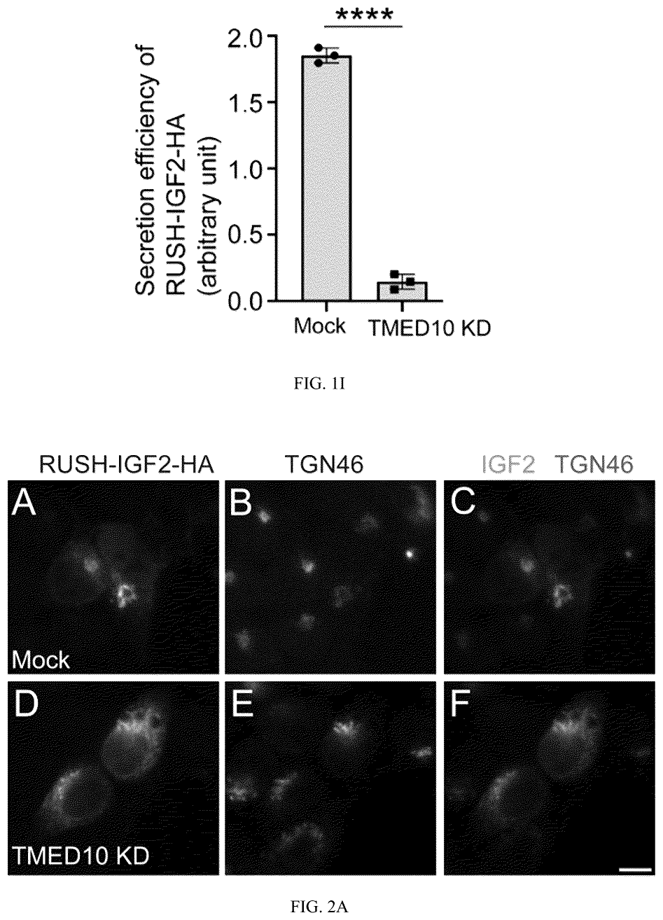

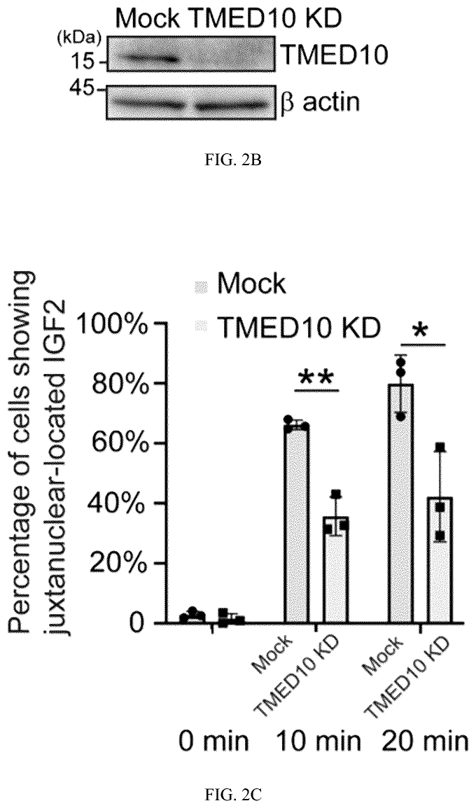

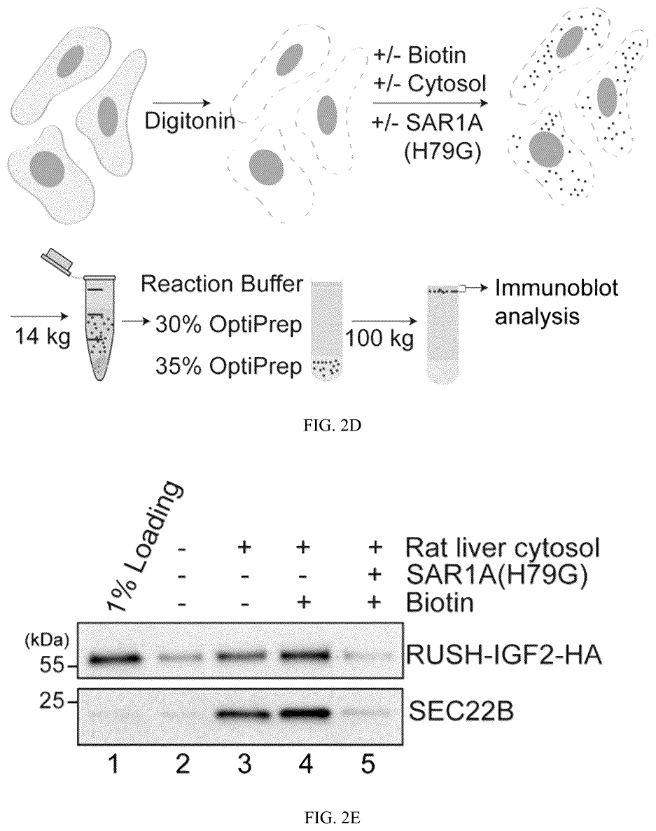

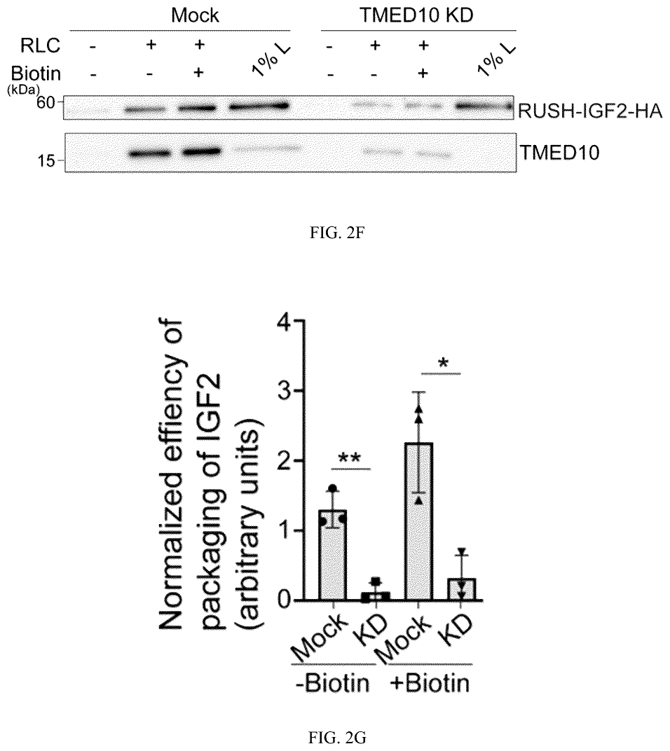

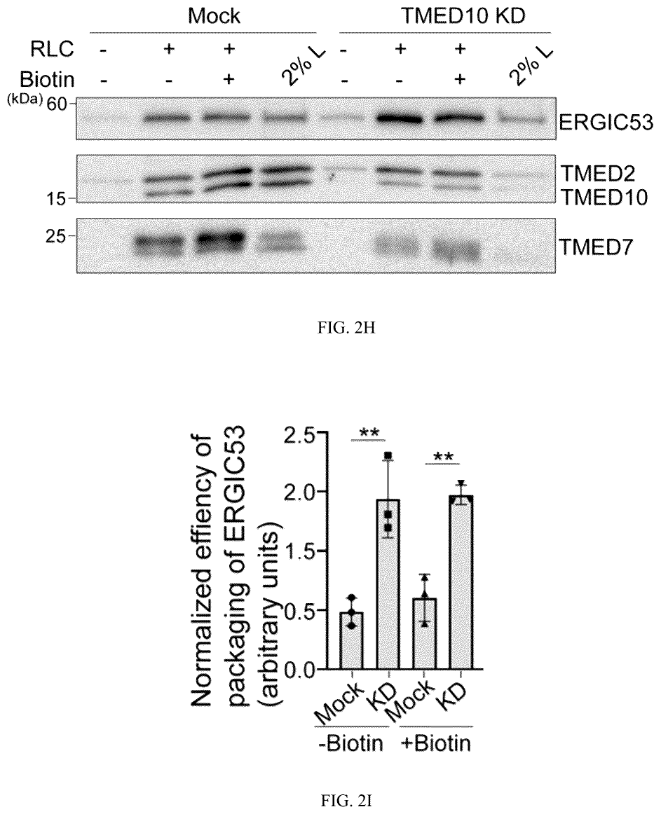

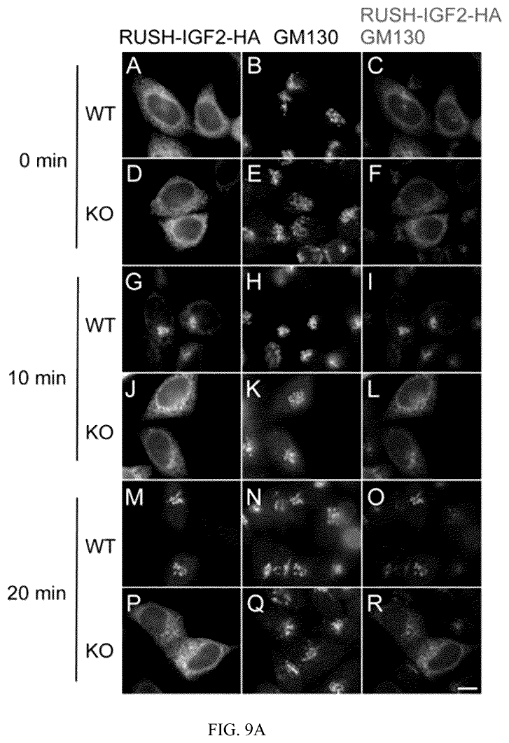

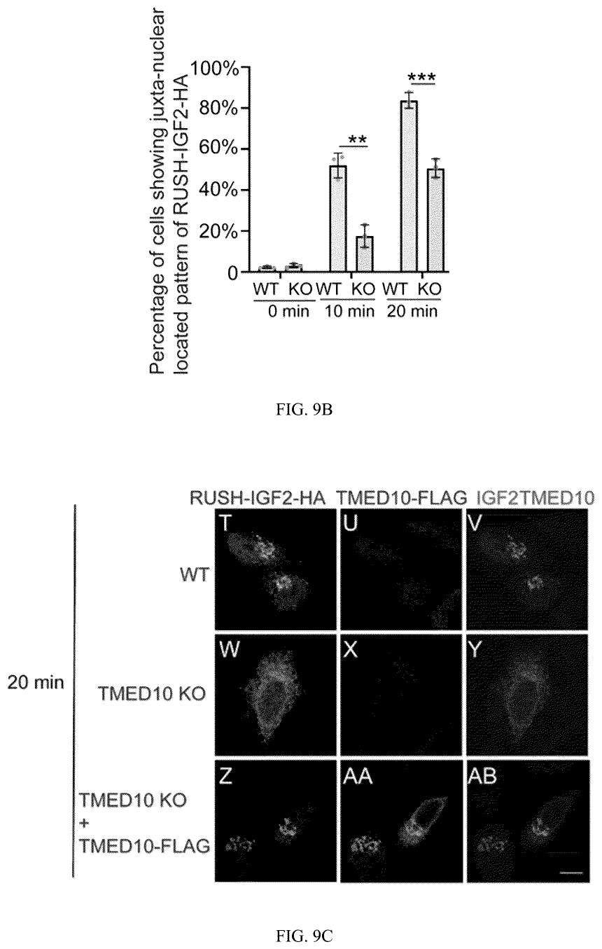

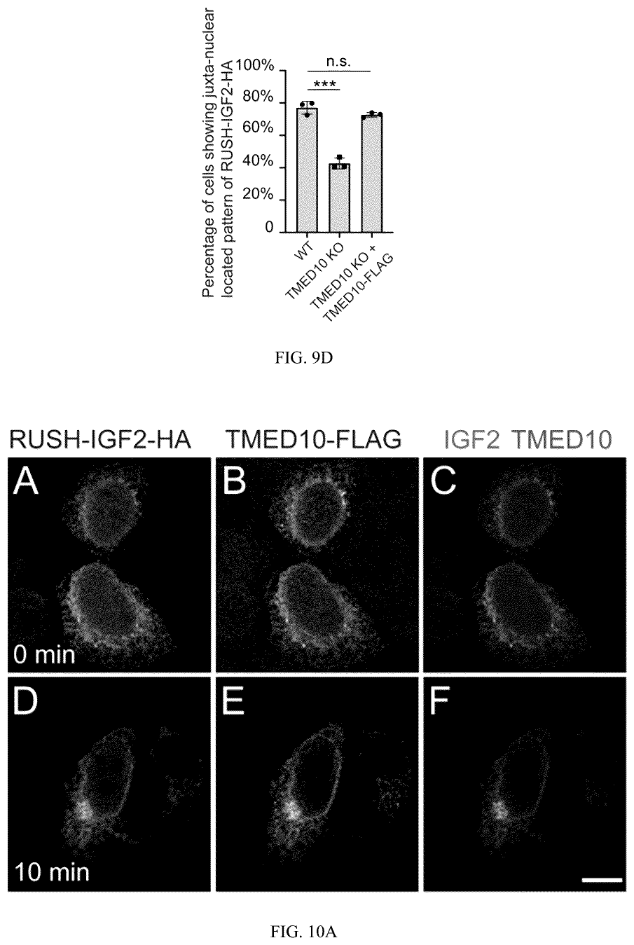

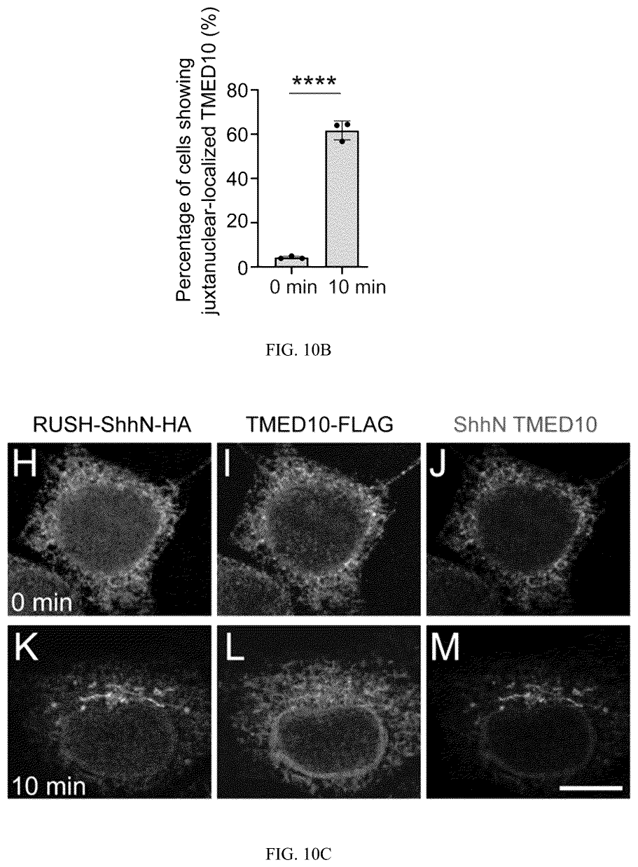

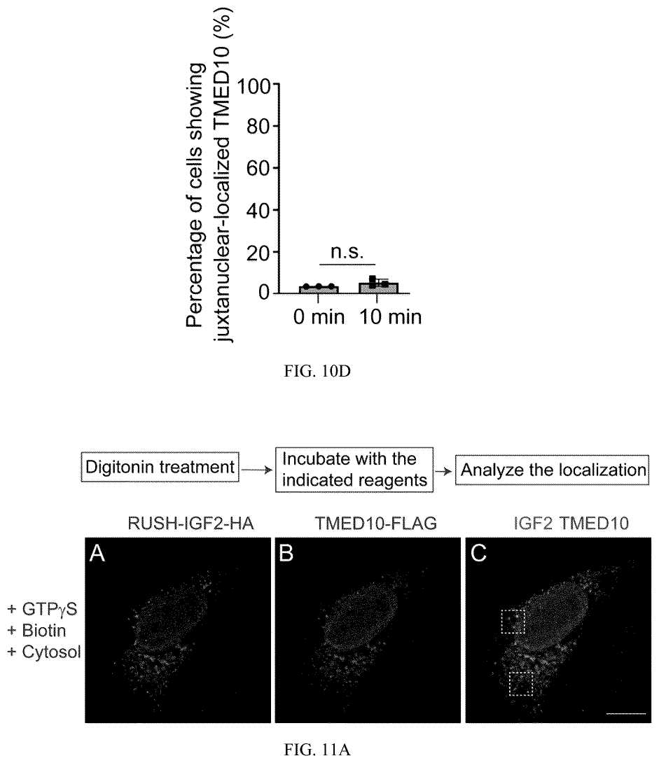

Next, we analyzed which step TMED10 is involved in the secretion of IGF2. We found that knockdown of TMED10 caused a defect in ER-to-Golgi trafficking of RUSH-IGF2-HA (FIGS. 2A and 2B and quantification in FIG. 2C). The defect was also observed in TMED10 KO cells (FIG. 9A and quantification in FIG. 9B). This defect was rescued by the expression of TMED10-FLAG in TMED10 KO cells (FIG. 9C and quantification in FIG. 9D), suggesting that TMED10 is important for ER-to-Golgi trafficking of RUSH-IGF2-HA. When TMED10-FLAG was co-expressed with RUSH-IGF2-HA in the absence of biotin, it was located at the ER in the majority of the TMED10- and IGF2-coexpressing cells (FIG. 10A; panels A-C). Under this condition, only around 4% of the co-expressing cells showed detectable localizations of TMED10-FLAG at the juxtanuclear area (FIG. 10B). After biotin treatment for 10 min, RUS-1-IGF2-HA was located at the juxta-nuclear Golgi area (FIG. 10A; panels D and F). Under this condition, TMED10-FLAG was colocalized with RUSH-IGF2-HA at the juxta-nuclear Golgi area in ˜60% of the co-expressing cells (FIG. 10A; panels D and F and quantification in FIG. 10B). The percentage of the co-expressing cells showing juxta-nuclear TMED10-FLAG was significantly higher in the presence of biotin than that detected in the absence of biotin (FIG. 10B). These analyses indicate that ER retention of IGF2 causes accumulations of TEMD10 at the ER. These analyses also indicate that TMED10 traffics together with RUSH-IGF2-HA from the ER to the Golgi. We then performed a similar analysis in cells co-expressing the RUSH construct of ShhN (RUSH-ShhN-HA) and TMED10-FLAG (FIGS. 10C and 10D). Our findings revealed that TMED10-FLAG did not co-traffic with RUSH-ShhN-HA from the ER to the Golgi following biotin treatment (FIG. 10D). These analyses indicate that TMED10 is co-transported with IGF2 but not ShhN from the ER to the Golgi.

We hypothesized that TMED10 functions as a cargo receptor to regulate packaging IGF2 into COPII vesicles. To test this hypothesis, we reconstituted the release of IGF2 into COPII vesicles using the vesicle formation assay [30, 31]. HEK293T cells transfected with RUSH-IGF2-HA were permeabilized by digitonin. After permeabilization, the semi-intact cells were washed with cold KOAc buffer to remove cytosolic proteins. The semi-intact cells were then incubated at 32° C. with GTP and an ATP regeneration system (ATPrS) in the presence or absence of biotin, rat liver cytosol (RLC) and a GTPase defective mutant of SAR1A, SAR1A (H79G) (FIG. 2D). The released vesicles after incubation were then isolated by centrifugation and analyzed by immunoblot (FIG. 2D). RUSH-IGF2-HA was detected in the vesicle fraction when the vesicle formation assay was performed in the presence of RLC (FIG. 2E, lane 3). We detected biotin-independent budding of RUSH-IGF2-HA suggesting the RUSH system does not block ER export. The abundance of IGF2 in the vesicle fraction was enhanced when the assay was performed in the presence of biotin likely due to the release from retrieval (FIG. 2E, compare lanes 3 and 4). Adding SAR1A (H79G) blocked the vesicular release of RUSH-IGF2-HA (FIG. 2E, compare lanes 4 and 5). These results indicate that this assay successfully reconstituted the release of IGF2 into COPII vesicles. Remarkably, knockdown of TMED10 caused a significant reduction in the abundance of RUSH-IGF2-HA in transport vesicles under both conditions (with or without biotin, FIGS. 2F and 2G), suggesting that TMED10 is important for packaging RUSH-IGF2 into COPII vesicles.

In yeast, ERV25 (the yeast homolog of human TMED10) forms a heteromeric complex with EMP24, ERP1 and ERP2 (the yeast homologs of human TMED2, 4, 7, respectively) [32]. Their protein levels are interdependent, and these proteins function in a cooperative manner [32]. In mammals, TMED10 exists in a hetero-oligomeric complex with TMED2, TMED7, and TMED9 [33, 34]. We then analyzed the efficiency of budding of two p24 family proteins, TMED2 and TMED7, and another cargo receptor, ERGIC53, in control cells and in cells knockdown of TMED10. Consistent with previous reports, the abundances of TMED7 and TMED2 in cell lysates were markedly reduced in TMED10 KD cells (FIG. 2H), indicating interdependence among TMED family proteins for their stability. Consequently, the abundance of these two cargo proteins in the vesicle fraction was also reduced (FIG. 2H). Interestingly, the packaging efficiency of ERGIC53 was found to be enhanced in TMED10 KD cells (FIGS. 2H and 2I).

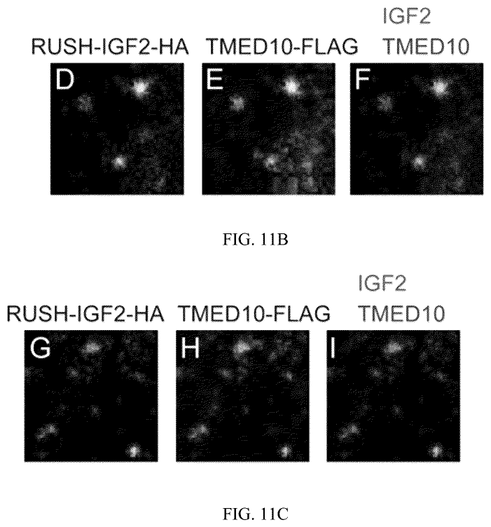

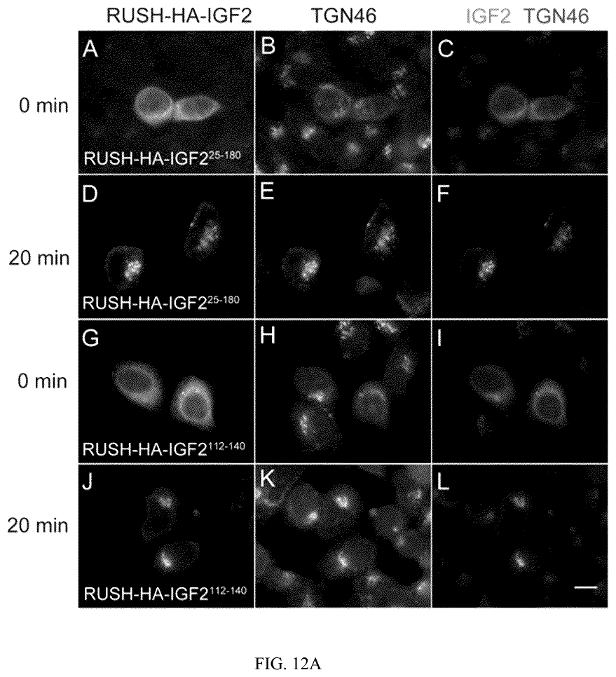

We next analyzed the colocalization between IGF2 and TMED10 utilizing a digitonin-permeabilized cell assay. We have previously demonstrated that this assay locks the ER export process at the sorting step [27], providing a convenient way to analyze the colocalization between cargo receptors and cargo molecules. Cells co-expressing RUSH-IGF2-HA and TMED10-FLAG were permeabilized by digitonin and washed with high salt buffer to remove the endogenous cytosolic proteins. Then, cells were incubated with rat liver cytosol, biotin and GTPγS for 15 min. After incubation, RUSH-IGF2-HA and TMED10-FLAG showed punctate localization patterns (FIG. 11A). Many IGF2 punctate structures overlapped with the punctate structures of TMED10 (FIG. 11A, magnified views in FIGS. 11B and 11C). This analysis indicates that IGF2 co-localizes with TMED10 upon exiting the ER.

### Example 3—Residues 112-140 in IGF2 are Important for ER-to-Golgi Trafficking of IGF2

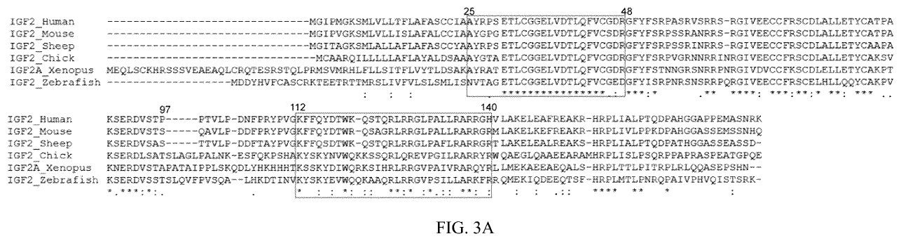

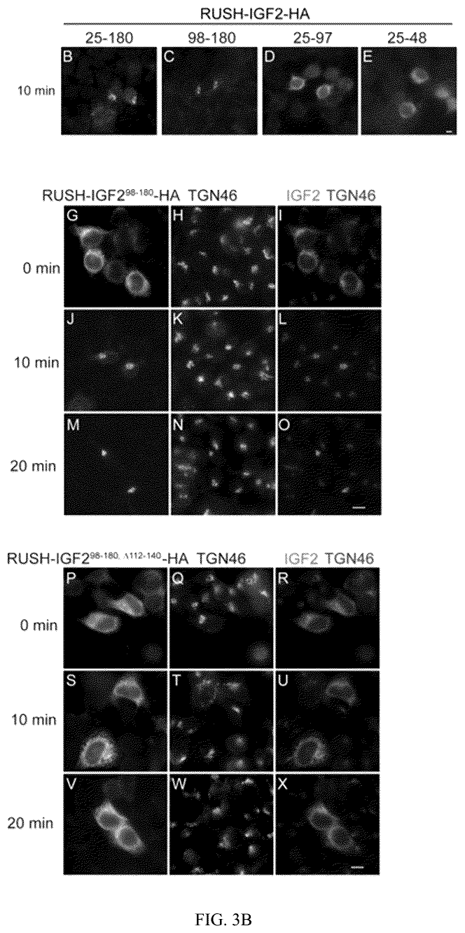

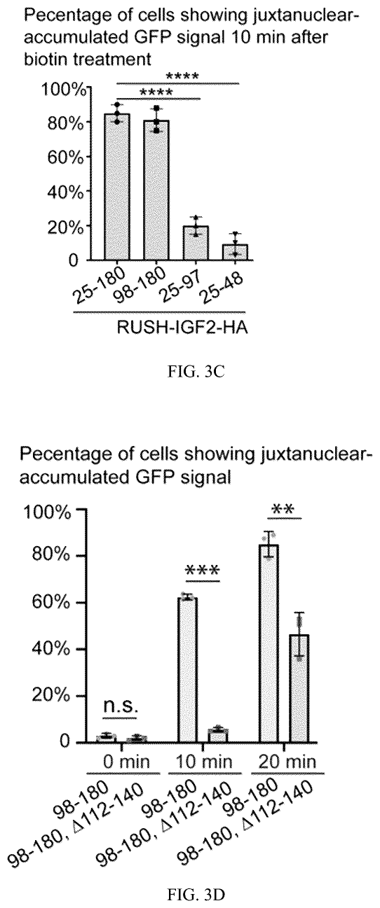

The next question we addressed is which motif of IGF2 is the major determinant for IGF2 trafficking and secretion. We performed sequence alignment of different IGF2 orthologues and synthesized different IGF2 truncated proteins; each contains one or several distinct highly conserved regions (FIG. 3A). Utilizing the RUSH assay, we tested ER-to-Golgi trafficking of these mutant constructs. Interestingly, we found that RUSH-IGF2²⁵⁻⁴⁸-HA was located at the ER in over 90% of cells after 10 min biotin treatment (FIGS. 3B and 3C). RUSH-IGF2²⁵⁻⁹⁷-HA also showed a defect of ER-to-Golgi trafficking (FIGS. 3B; panel D, and 3C). In contrast, RUSH-IGF2⁹⁸⁻¹⁸⁰-HA localized at the Golgi apparatus in around 80% of the cells after biotin treatment for 10 min (FIGS. 3B; panel C, and 3C), suggesting that IGF2⁹⁸⁻¹⁸⁰ is the critical part of IGF2 trafficking from the ER to the Golgi.

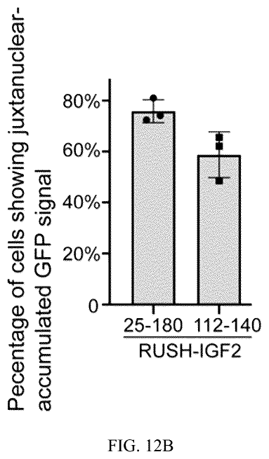

Sequence alignment indicates that the residues between position 112 and 140 of IGF2 are conserved across species (FIG. 3A, highlighted in the green box). To test whether these residues are important for ER export of IGF2, we generated a RUSH construct of IGF2⁹⁸⁻¹⁸⁰ fragment depleted these residues (RUSH-IGF2^{98-180, Δ112-140}-HA). Upon biotin treatment for 10 min or 20 min, RUSH-IGF2⁹⁸⁻¹⁸⁰-HA showed juxtanuclear localization in the majority of cells (FIGS. 3B; panels G-O, and 3D). In contrast, the majority of cells expressing RUSH-IGF2^{98-180, Δ112-140}-HA showed an ER pattern (FIGS. 3B; panels P-X, and 3D). Further analyses indicates that residues 112 to 140 in IGF2 are sufficient for SBP-EGFP to be delivered from the ER to the Golgi with an efficiency that is similar to full length IGF2 (FIGS. 12A and 12B). In summary, these observations show that IGF2¹¹²⁻⁰⁴⁰ is the ER-to-Golgi transport motif of IGF2.

### Example 4—IGF2 Interacts with the Gold Domain of TMED10 and this Interaction Depends on Residues 112-140 in IGF2

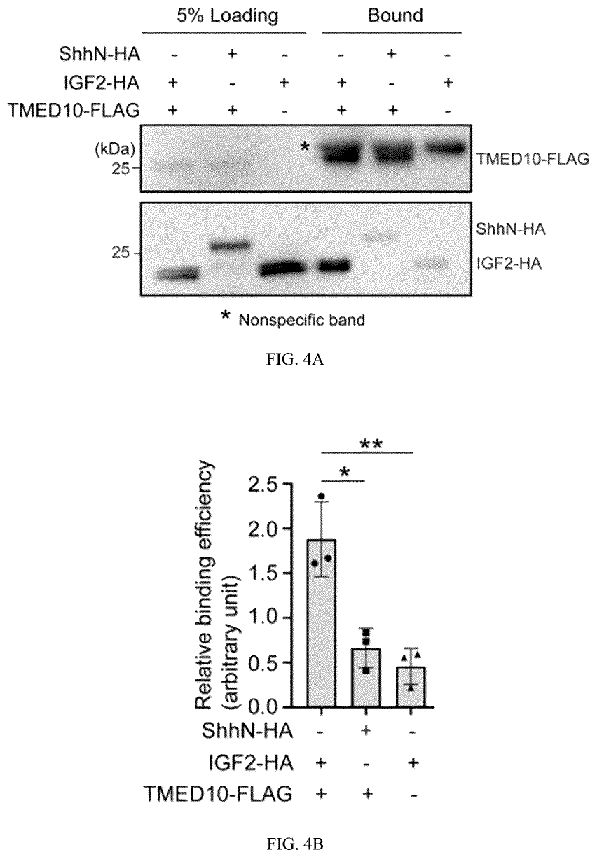

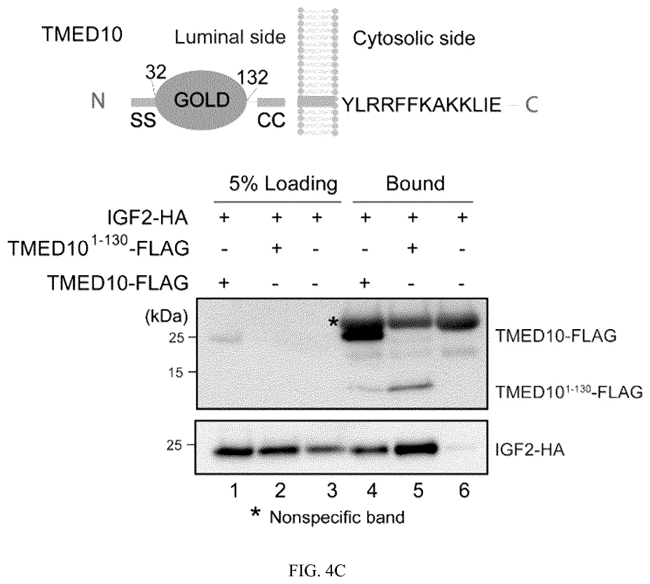

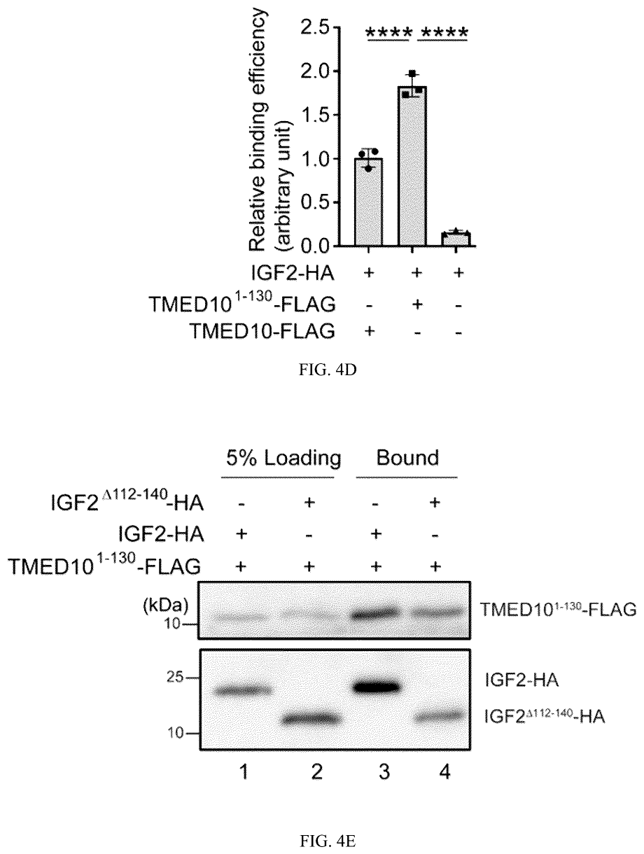

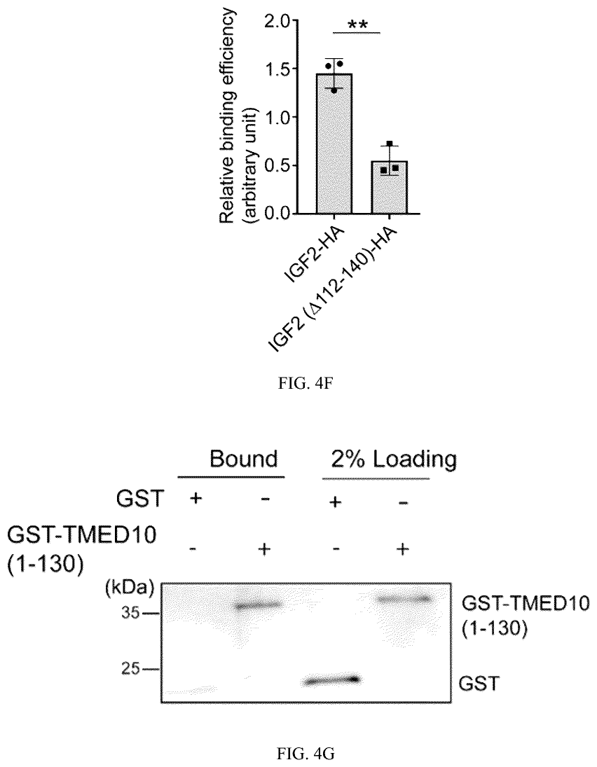

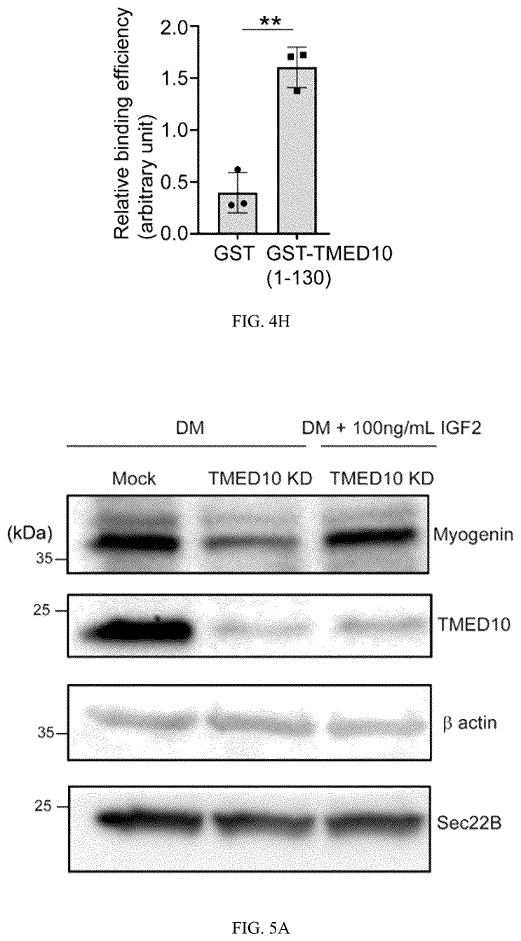

We then performed co-immunoprecipitation (co-IP) experiments using HEK293T cells co-transfected with plasmids encoding FLAG-tagged TMED10 (TMED10-FLAG) and HA-tagged IGF2 or ShhN (IGF2-HA or ShhN-HA). A crosslinker DSP was used in the co-IP experiments to stabilize the interaction. The co-IP assay revealed that IGF2-HA bound TMED10-FLAG in cell lysates (FIG. 4A). The percentage of IGF2-HA that bound to TMED10-FLAG was significantly higher than the percentage of ShhN-HA in cell lysates that bound to TMED10 (FIGS. 4A and 4B), indicating TMED10 specifically interacts with IGF2.

TMED family proteins have highly conserved structures. The luminal part of TMED proteins consists of the signal sequence (SS), the Golgi dynamics (GOLD) domain and the coiled-coil (CC) region [35, 36](FIG. 4C). The cytosolic portion of many TMED proteins contain two C-terminal hydrophobic residues that promote ER export [37] and dilysine motifs (KK) that are important for ER retrieval [38]. TMED10, while it possesses a dilysine motif within its cytosolic domain, lacks hydrophobic residues at its C-terminus. As TMED10 forms a complex with other proteins from the TMED10 family, TMED10 might be enriched into COPII vesicles through the ER export motif found in other members of the TMED family. The GOLD domain is implicated in recognizing cargo proteins [39]. We then generated FLAG-tagged TMED10¹⁻¹³⁰, which contains the SS motif and the GOLD domain, to test whether the GOLD domain is sufficient for the interaction. Strikingly, the abundance of IGF2-HA that bound to TMED10¹⁻¹³⁰-FLAG was significantly higher than that bound to the full-length TMED10-FLAG (FIG. 4C, compare lanes 4 and 5, and FIG. 4D), suggesting that TMED10 binds IGF2 through its luminal GOLD domain.

Since the ER-to-Golgi transport of IGF2 depends on its residues between 112 and 140, we next tested whether this motif is important for the IGF2-TMED10 interaction. We found that depleting this motif significantly reduced the abundance of IGF2-HA that bound to TMED10-FLAG (FIG. 4E, compare lanes 3 and 4, and FIG. 4F). We then performed a peptide binding assay to study whether this interaction is direct. Synthesized peptides corresponding to the 112-140 residues of IGF2 (IGF2¹¹²⁻¹⁴⁰) were covalently linked to beads. The beads were then incubated with purified GST or GST-tagged TMED10 GOLD domain (GST-TMED10¹⁻¹³⁰). The result shows that the abundance of GST-TMED10¹⁻¹³⁰ that bound to IGF2¹¹²⁻¹⁴⁰ was significantly higher than the abundance of GST that bound to the peptides (FIGS. 4G and 4H), suggesting the ER export motif of IGF2 interacts with the GOLD domain of TMED10 directly.

### Example 5—TMED10 is Important for the Secretion of IGF2 from C2C12 Cells for Muscle Stem Cell Differentiation

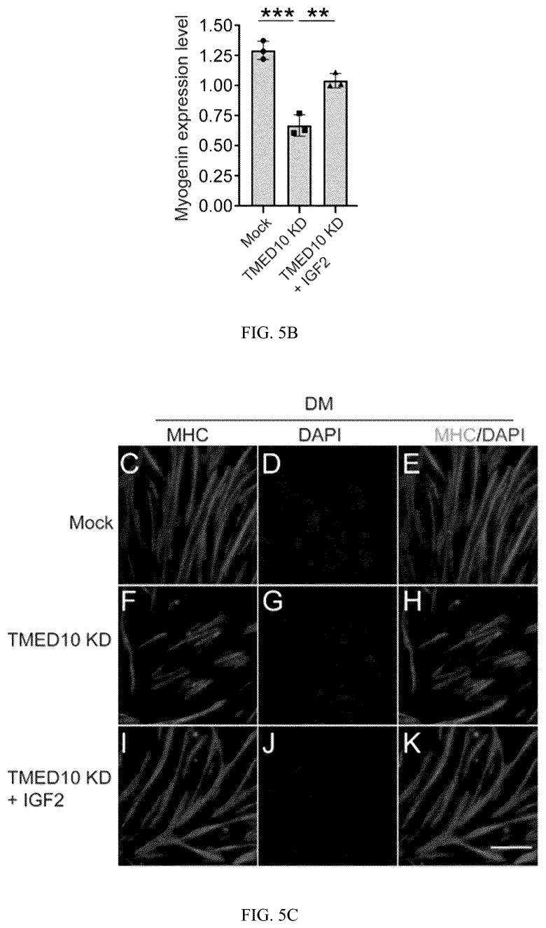

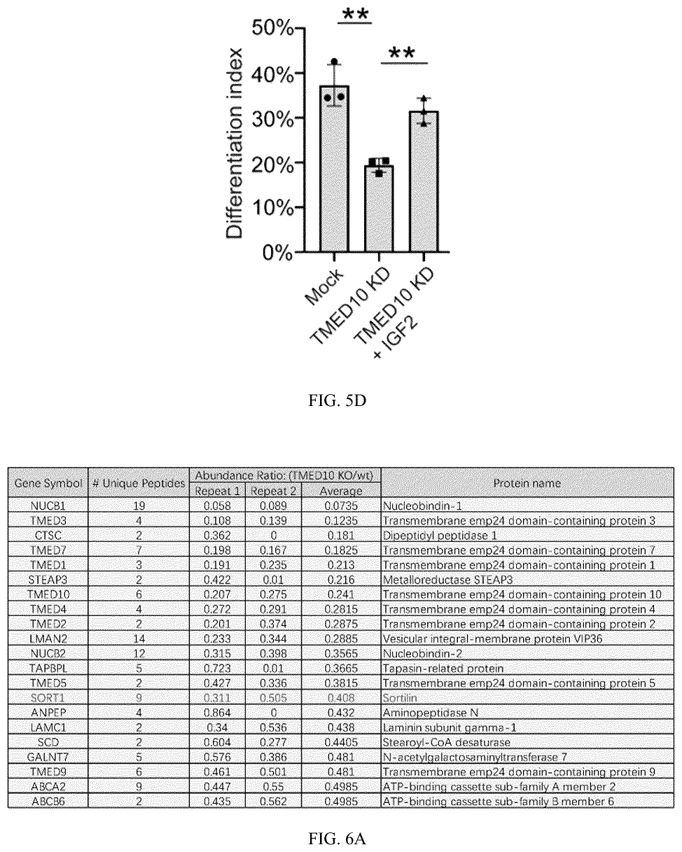

We then tested whether TMED10 is important for the secretion of IGF2 from mouse C2C12 myoblasts. We collected the medium incubated with C2C12 cells transfected with control siRNA or siRNA against TMED10. The proteins in the medium were TCA precipitated and then analyzed by label free quantitative mass spectrometry to compare the abundances of proteins detected in the medium from the two experimental groups. The abundance of IGF2 in the medium was greatly reduced in TMED10 knockdown cells compared to control cells in both of the replicated experiments. In addition to IGF2, we identified 52 secretory proteins whose abundances exhibited a least more than 1.9-fold higher in the medium of control cells than those detected in the medium of TMED10 knockdown cells in each of the replicated experiments. Expression of the myoblast differentiation marker, Myogenin, was significantly reduced in TMED10 knockdown cells incubated with the differentiation medium (DM) when compared to the control cells incubated at the same condition (FIG. 5A). Adding purified IGF2 into the differentiation medium rescues the expression of Myogenin in TMED10 knockdown cells (FIG. 5A and quantification in FIG. 5B). These results indicate that TMED10 plays an important role in myoblast differentiation by functioning as a cargo receptor to enrich IGF2 into COPII vesicles, a process that is crucial for the secretion of IGF2. To further analyze the rescue effects, we analyzed the myotube formation of C2C12 cells by staining the myosin heavy chain (MHC), a marker protein of the myotubes. Consistent with our western blot result, the myotube became much shorter and thinner after knockdown of TMED10 (FIG. 5C). After incubation with purified IGF2, myotube formation was rescued, and myotubes became longer and thicker (FIG. 5C). We then quantified the differentiation index after the C2C12 differentiation assay. The differentiation index was significantly reduced in TMED10 KD cells and this defect was recused by purified IGF2 (FIG. 5D). These analyses indicate that TMED10 regulates C2C12 differentiation through an autocrine manner. Taken together, we revealed that TMED10 is important for packaging IGF2 into COPII vesicles to deliver IGF2 from the ER to the Golgi, and this step is critical for myoblast differentiation.

### Example 6—Sortilin is Another Cargo Client of TMED10

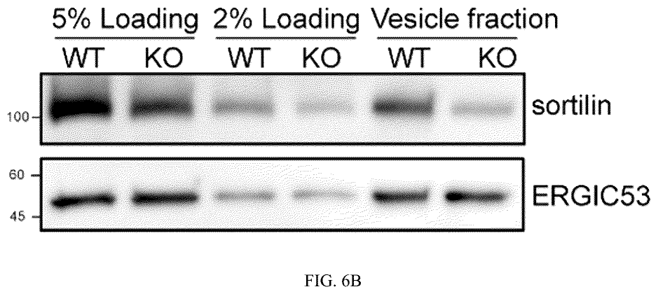

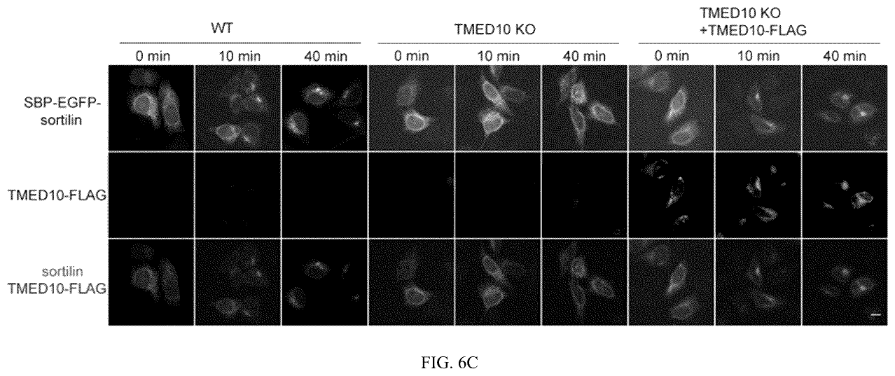

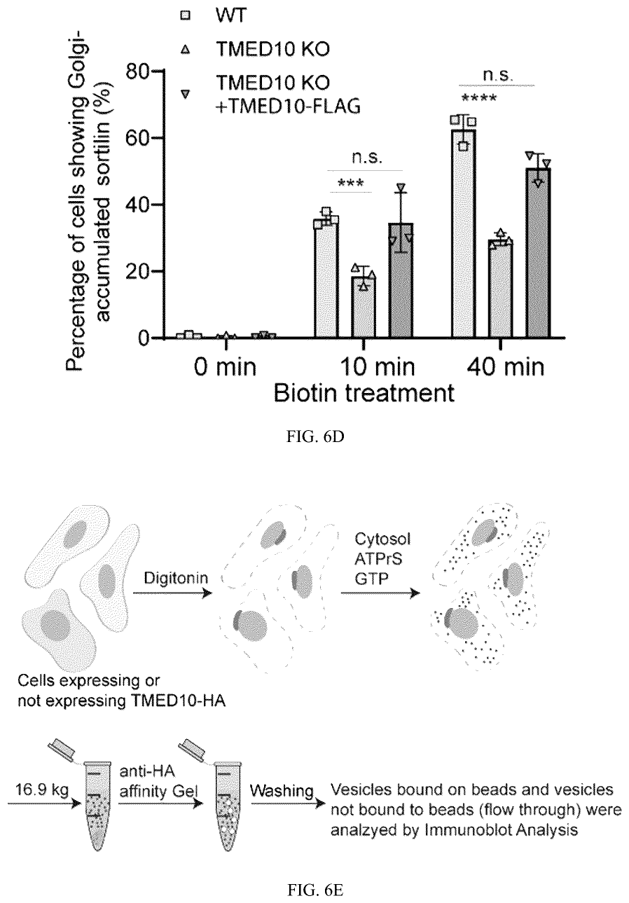

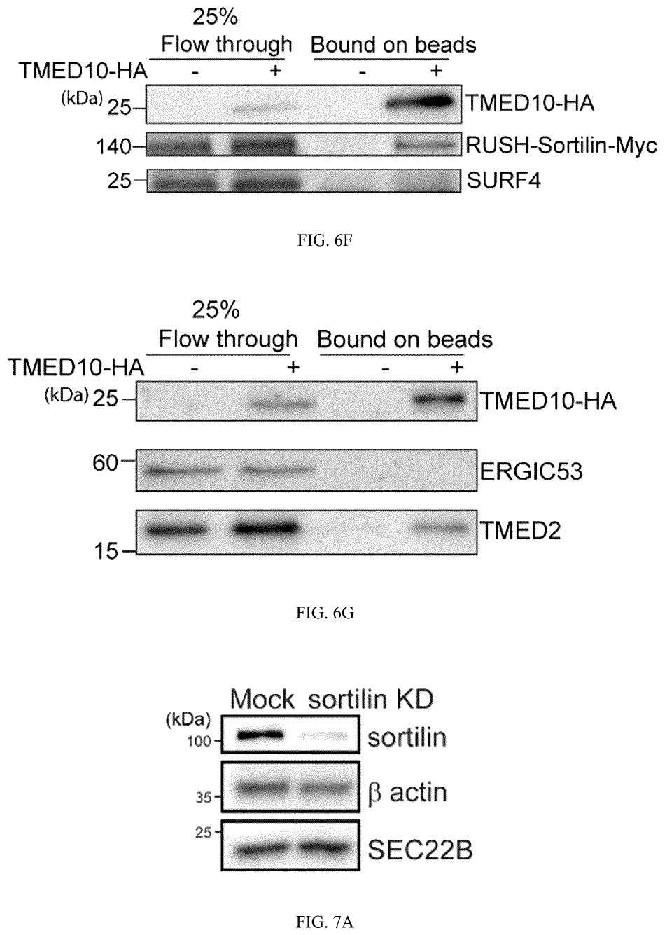

Next, we sought to identify other cargo proteins that depends on TMED10 to be enriched into transport vesicles. We have previously developed a vesicle formation assay in combination with a label-free quantitative mass spectrometry approach and this approach revealed the cargo clients of two ER cargo receptors, ERGIC53 and SURF4 [30]. We utilized a similar approach to uncover the cargo clients of TMED10. A large-scale vesicle formation assays were performed using donor membranes provided by wild type (WT) or TMED10 KO HeLa cells. A label-free quantitative mass spectrometry analysis was then conducted to compare the protein profiling of vesicles produced from these two experimental groups (the WT group and TMED10 KO group). We detected peptides that match TMED10 in the vesicle fraction generated by the TMED10 KO cells. A possible explanation is that a negligible quantity of TMED10 may persist in rat liver cytosol prepared from rat livers, which may associate with vesicles after the vesicle formation assay. Although this residual amount of proteins is not detectable through immunoblotting, it may be detected using mass spectrometry that has the capacity to detect proteins in the low picogram range. We found that the abundance of a series of transmembrane proteins is greatly reduced in the vesicle fraction in TMED10 KO group based on two biological repeats (FIG. 6A, average fold change of TMED10 KO/WT<0.5). These identified transmembrane proteins including several p24 family proteins: TMED1, TMED2, TMED3, TMED4, TMED5, TMED7, and TMED9 (FIG. 6A). This decrease is presumably caused by the degradation of these TMED proteins induced by the depletion of TMED10, thereby reducing their presence not only within the cells but also in the vesicle fraction.

In addition, we identified a Golgi- and plasma-membrane localized transmembrane protein, sortilin, that depends on TMED10 to be enriched into transport vesicles (FIG. 6A, highlighted in red, FIG. 6B). Sortilin is a single-pass transmembrane protein belonging to the vacuolar protein sorting 10 protein (Vps10p) family [15]. Here, we discovered that TMED10 functions as a cargo receptor that mediates the ER-to-Golgi transport of sortilin.

The total level of sortilin was greatly decreased in both cell lysates and vesicle fraction of the KO group (FIG. 6B). To investigate whether TMED10 is important for the ER-to-Golgi trafficking of newly synthesized sortilin, we generated a RUSH construct of sortilin and performed the RUSH transport assay in WT or TMED10 KO HeLa cells. We found that the ER-to-Golgi transport of sortilin was strongly impaired in KO cells (FIGS. 6C and 6D). In fact, after biotin treatment for 40 minutes, the majority of sortilin was trapped at the ER. This defect was rescued by transfecting TMED10-FLAG in KO cells (FIGS. 6C and 6D). These results indicate that TMED10 is also essential for ER-to-Golgi trafficking of sortilin. We then performed the vesicle formation assay and immunoisolated vesicles enriched with TMED10-HA (FIG. 6E). Our findings revealed that these isolated vesicles contained RUSH-sortilin-Myc and TMED2 (FIGS. 6F and 6G), but not SURF4 and ERGIC53 (FIGS. 6F and 6G). This analysis indicates that sortilin and TMED2 reside in the same vesicles as TMED10, unlike SURF4 and ERGIC53.

### Example 7—Sortilin Regulates TGN Export of IGF2

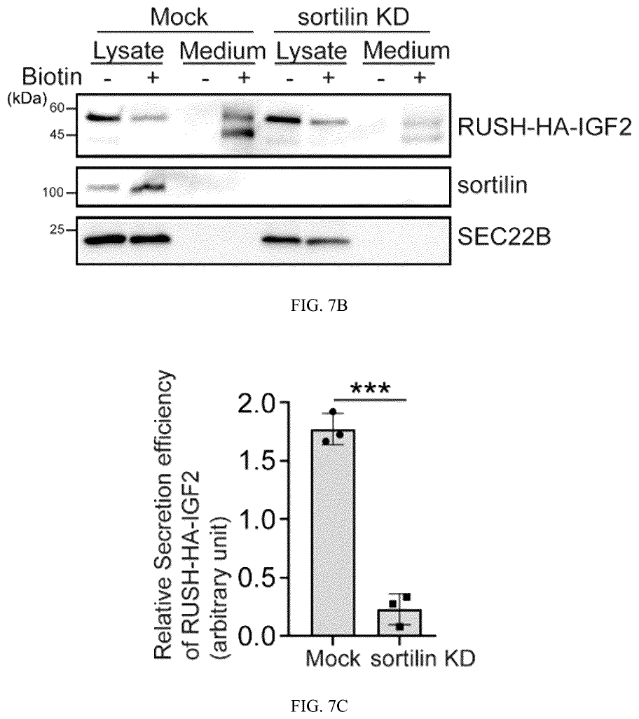

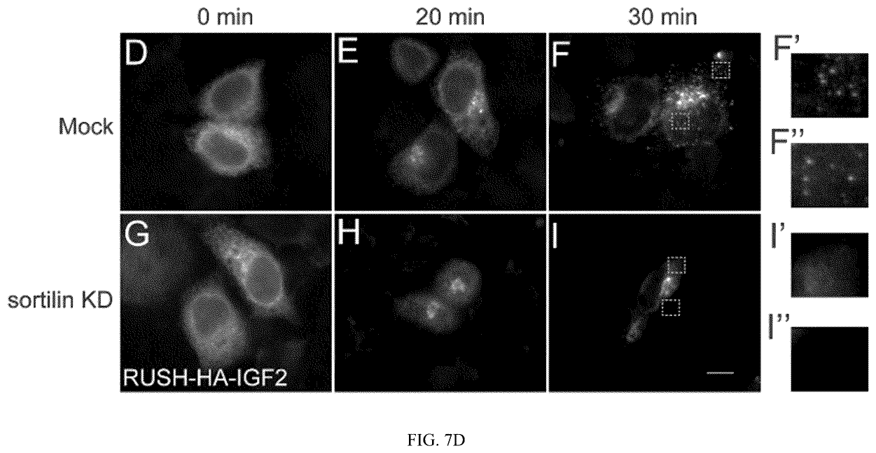

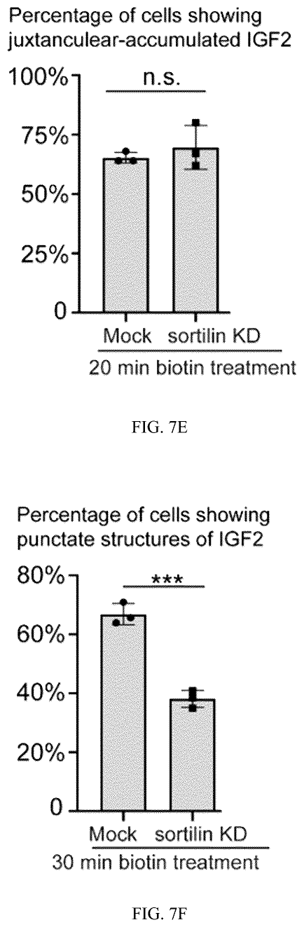

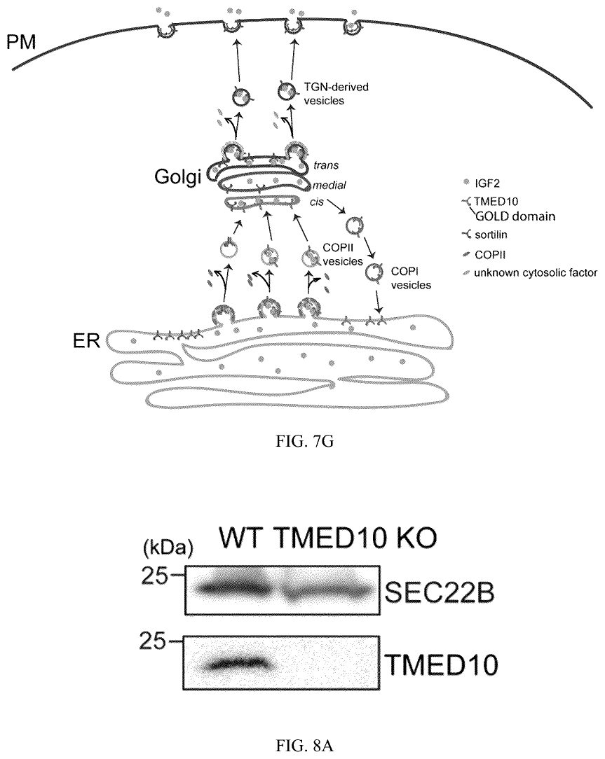

Our previous analyses indicate that ER-to-Golgi trafficking of IGF2 and sortilin is mediated by TMED10. Sortilin mediates insulin-dependent glucose transport in myocytes and is crucial for myogenesis [19, 40]. Interestingly, we found that knockdown of sortilin significantly reduced the efficiency of RUSH-HA-IGF2 secretion after biotin treatment (FIGS. 7A-7C), indicating that sortilin plays a crucial role in IGF2 secretion. To determine whether sortilin is required for ER-to-Golgi trafficking or TGN-to-PM transport of IGF2, we analyzed the RUSH-HA-IGF2 trafficking in sortilin KD HeLa cells at different time points after biotin treatment. The percentage of cells showing juxta-nuclear localized RUSH-HA-IGF2 was similar in Mock and sortilin KD group after biotin treatment for 20 min (FIGS. 7D, and 7E), suggesting that knockdown of sortilin did not affect the ER-to-Golgi trafficking of RUSH-HA-IGF2. RUSH-HA-IGF2 showed punctate structures in the cell periphery in over 60% of cells after biotin treatment for 30 min. We hypothesize that these punctate structures are TGN-derived vesicles enriched with RUSH-HA-IGF2. The percentage of cells showing punctate structures of RUSH-HA-IGF2 was significantly decreased in sortilin KD group compared to the control group (FIGS. 7D, and 7F). These analyses demonstrated that sortilin is important for TGN export but not ER export of IGF2.

We found that two transmembrane proteins, TMED10 and sortilin, cooperatively mediate IGF2 secretion along the secretory pathway. Based on our study, we propose that the secretion of IGF2 is achieved by several steps (FIG. 7G). Firstly, the correctly folded ER-localized pro-IGF2 is captured into COPII vesicles by the direct interaction between the IGF2¹¹²⁻¹⁴⁰ motif and TMED10 GOLD domain. Secondly, vesicles containing pro-IGF2 and TMED10 are delivered to the Golgi apparatus where the pro-IGF2 receives O-glycosylation modifications and cleavage. At the Golgi, TMED10 is disassociated from IGF2 and is recycled to the ER by COPI vesicles. In addition, TMED10 also mediates ER export of sortilin. After reaching TGN, sortilin regulates TGN-to-cell surface delivery of IGF2 (FIG. 7G). Therefore, sortilin is important for the post-Golgi trafficking of IGF2. These findings support a key role for TMED10 in indirectly mediating TGN export of IGF2 by regulating the ER-to-Golgi trafficking of sortilin.

### Discussion

TMED10 is a member of the p24 family. A null mutation in TMED10 results in early embryonic lethality in mice [41].

The inactivation of one allele of TMED10 in mice causes dilation of Golgi cisternae [41] and knockdown of TMED9 in HeLa cells caused dispersal of the Golgi [42]. TMED10 negatively regulates autophagy, and the expression of TMED10 is reduced in Alzheimer's disease patients [43].

The yeast homologue of TMED10 forms a complex with the yeast homologue of TMED2 and this complex regulates ER-to-Golgi transport of a GPI-anchored protein, Gas1p [44]. In mammalian cells, TMED10 is also shown to regulate surface delivery of a GPI-anchored proteins [45]. GTP-bound form of Rab21 was shown to interact with TMED10 and regulate localizations of TMED10 at the Golgi [46]. Immunoprecipitation results revealed that both TMED10 and TMED2 showed a preference to interact with Sec24C and Sec24D, indicating Sec24C and Sec24D are two Sec24 isoforms involved in TMED10 mediated protein trafficking at the ER [47]. The majority of p24 family proteins are primarily located within the luminal side of organelle membranes. Their asymmetric nature imposes a curvature that is opposite to the curvature needed for vesicle budding, thereby changing the physical characteristics of membranes [48]. It has been shown that the scaffolding function of the cargo adaptor Lst1p, the yeast homologue of Sec24, and the outer COPII coat Sec13p are essential to counter the resistance caused by the p24 proteins and facilitate vesicle formation at the ER [48, 49].

Upon reaching the Golgi, cargo molecules dissociate from their clients. We have previously demonstrated that proteoglycans compete with SURF4 to interact with Shh at the Golgi thereby causing SURF4 to be dissociated from its client [27]. P24 family proteins have been shown to interact with the remodeled GPI-APs to enrich them into COPII vesicles [50, 51]. This interaction is pH-dependent, suggesting that they may be dissociated from each other at the Golgi due to pH changes [50]. Moreover, p24 proteins have been demonstrated to retrieve escaped, unremodeled GPI-anchored proteins from the Golgi, returning them to the ER within COPI vesicles [51]. This suggests that p24 proteins play a crucial role in monitoring anchor remodeling to ensure accurate trafficking of GPI-APs [51]. The transmembrane domain of TMED2 (but not TMED10) has been found to interact specifically with a sphingomyelin, SM18. This interaction facilitates efficient retrograde COPI-dependent trafficking and is implicated to modulate the equilibrium between monomeric and oligomeric states of TMED2 [52].

Intriguingly, TMED10 is also shown to function as a protein channel to mediate the unconventional protein secretion (UPS) of a group of leaderless proteins including IL1β, IL-1α, HSPB5, Tau and Annexin A1 [53]. Unlike the TMED10 mediated conventional secretory pathway, TMED10-channeled UPS requires direct or indirect interactions between UPS cargoes and TMED10 C-terminal Tail [36]. In the unconventional secretory pathway, TMED10 is triggered by the production of UPS cargoes to form a higher order mono-oligomer which stabilizes the TMED10 protein channel on ERGIC membranes for UPS protein translocation [36]. If oligomeric form of TMED10 forms a channel, the pore of this channel would be highly hydrophobic as the transmembrane residues of TMED10 are predominantly hydrophobic. This creates an energy barrier for the passage of IL1β and other unconventional cargo proteins. In addition, IL1β and other unconventional secretory proteins are efficiently secreted upon infection-induced permeabilization of the plasma membrane [54], suggesting a substantial accumulation of these cargo molecules within the cytoplasm of immune cells. Deleting the GOLD domain abolished TMED10 mono-oligomerization, indicating that the integrity of the GOLD domain is crucial for the mono-oligomerization of TMED10 [36]. The direct interaction between IGF2 and TMED10 GOLD domain may interfere with TMED10 mono-oligomerization. In addition, the quantitative mass spectrometry analysis indicates that TMED1, 2, 3, 7 and 9 showed a large decrease in TMED10 KO vesicles (FIG. 6A). Thus, we hypothesized that TMED10 and other p24 family proteins form hetero-oligomers to package IGF2 into COPII vesicles.

In addition to the selective capture mechanism, bulk flow is another approach that exports soluble or membrane-associated proteins from the ER [13]. Export by bulk flow does not rely on cargo receptors or export motifs on cargoes. Instead, proteins exported by bulk flow were packaged into COPII vesicles by default. Utilizing the RUSH assay, we found that RUSH-IGF2⁹⁸⁻¹⁸⁰-HA without the 112-140 aa motif showed a kinetic delay in ER-to-Golgi transport (FIGS. 3B-3D). In contrast, fusing the SBP-EGFP tag with IGF2¹¹²⁻¹⁴¹ motif efficiently brings SBP-EGFP to the Golgi (FIG. 12A; panels J-L), indicating that bulk flow is not an efficient approach in mediating ER-to-Golgi trafficking of IGF2. Cross-linking and peptide binding experiments revealed a novel direct interaction between TMED10 GOLD domain and the IGF2¹¹²⁻¹⁴⁰ motif, suggesting that TMED10 directly mediates the ER export of IGF2.

In summary, our work provides novel insights into the molecular machinery that mediates the trafficking of IGF2 along the secretory pathway to perform its physiological functions. Dysregulation of IGF2 activities is a candidate risk factor for tumorigenesis and is related to multiple disorders, such as Beckwith-Wiedemann syndrome, Silver-Russell syndrome and Doege-Potter syndrome [55-57]. The uncovered cellular factors and protein interactions that are important for the secretion of IGF2 provide novel therapeutic targets to downregulate IGF2 signaling by blocking IGF2 secretion [58].

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### Exemplary Embodiments

Embodiment 1. A pharmaceutical composition for inhibiting IGF2 signaling, the composition comprising an inhibitor of IGF2-TMED10 interaction in a cell and one or more pharmaceutical carriers or excipients.

Embodiment 2. The composition of embodiment 1, wherein the inhibitor comprises: (a) a human IGF2 polypeptide that comprises residues 112-140 of the human IGF2 sequence (SEQ ID NO:1); (b) a human TMED10 polypeptide that comprises residues 1-130 of the human TMED10 sequence; (c) a human TMED10 polypeptide that comprises residues 32-132 of the human TMED10 sequence; (d) a small molecule; (e) a covalent inhibitor: (f) an antibody; or (g) a genome editing tool.

Embodiment 3. A method of treating a subject having abnormal IGF2 signaling, the method comprising: (a) obtaining a pharmaceutical composition comprising an inhibitor of IGF2-TMED10 interaction in a cell and optionally one or more pharmaceutical carriers or excipients; and (b) administering an effective amount of the composition to the subject, wherein the inhibitor comprises a human IGF2 polypeptide comprising residues 112-140 of IGF2 sequence, a human TMED10 polypeptide that comprises residues 1-130 of the human TMED10 sequence, a human TMED10 polypeptide that comprises residues 32-132 of the human TMED10 sequence, a small molecule, a covalent inhibitor, an antibody; or a genome editing tool.

Embodiment 4. The method of any preceding embodiment, wherein the subject is a mammal.

Embodiment 5. The method of embodiment 4, wherein the mammal is a human.

Embodiment 6. The method of any preceding embodiment, wherein the subject is affected by Beckwith-Wiedemann syndrome, Silver-Russell syndrome, or Doege-Potter syndrome.

Embodiment 7. The method of any preceding embodiment, wherein the secretion of IGF2 in the cell is blocked by the inhibitor.

Embodiment 8. A method of treating cancer in a subject in need thereof, the method comprising: (a) providing a pharmaceutical composition comprising an inhibitor of the IGF2-TMED10 interaction in a cell and optionally one or more pharmaceutical carriers or excipients; and (b) administering an effective amount of the composition to a subject affected by a cancer, and optionally one or more pharmaceutical carriers or excipients, wherein the inhibitor comprises a human IGF2 polypeptide comprising residues 112-140 of IGF2 sequence, a human TMED10 polypeptide that comprises residues 1-130 of the human TMED10 sequence, a human TMED10 polypeptide that comprises residues 32-132 of the human TMED10 sequence, a small molecule, a covalent inhibitor, an antibody; or a genome editing tool.

Embodiment 9. The method of any preceding embodiment, wherein the subject is a mammal.

Embodiment 10. The method of embodiment 9, wherein the mammal is a human.

Embodiment 11. The method of any preceding embodiment, wherein the secretion of IGF2 in the cell is blocked by the inhibitor.

Embodiment 12. The method of any preceding embodiment, wherein the composition is administered to the subject via injection.

Embodiment 13. The method of any preceding embodiment, wherein the inhibitor comprises a CRISPR/CAS9 (RNA-guided targeted) genome editing tool, wherein the CRISPR/CAS9 editing tool is used to mutate the IGF2 sequence that codifies for residues 112-140 of IGF2.

Embodiment 14. The method of any preceding embodiment, wherein the inhibitor comprises a CRISPR/CAS9 (RNA-guided targeted) genome editing tool, wherein the CRISPR/CAS9 editing tool is used to mutate the human TMED10 sequence that codifies for residues 1-130 of the human TMED10 sequence, wherein the mutated TMED10 blocks the TMED10 binding to IGF2.

Embodiment 15. The method of embodiment 8, wherein the inhibitor comprises a CRISPR/CAS9 (RNA-guided targeted) genome editing tool, wherein the CRISPR/CAS9 editing tool is used to mutate the IGF2 sequence that codifies for residues 112-140 of IGF2.

Embodiment 16. The method of embodiment 8, wherein the inhibitor comprises a CRISPR/CAS9 (RNA-guided targeted) genome editing tool, wherein the CRISPR/CAS9 editing tool is used to mutate the human TMED10 sequence that codifies for residues 1-130 of the human TMED10 sequence, wherein the mutated TMED10 blocks the TMED10 binding to IGF2.

### REFERENCES

1. W. Chao, P. A. D'Amore, IGF2: epigenetic regulation and role in development and disease. Cytokine Growth Factor Rev 19, 111-120 (2008).
2. J. R. Florini, D. Z. Ewton, S. L. Roof, Insulin-like growth factor-I stimulates terminal myogenic differentiation by induction of myogenin gene expression. Mol Endocrinol 5, 718-724 (1991).
3. J. R. Florini et al., “Spontaneous” differentiation of skeletal myoblasts is dependent upon autocrine secretion of insulin-like growth factor-II. J Biol Chem 266, 15917-15923 (1991).
4. Y. Ge, Y. Sun, J. Chen, IGF-II is regulated by microRNA-125b in skeletal myogenesis. J Cell Biol 192, 69-81 (2011).
5. Y. Guo, G. Zanetti, R. Schekman, A novel GTP-binding protein-adaptor protein complex responsible for export of Vang12 from the trans Golgi network. Elife 2, e00160 (2013).
6. X. Tang et al., Molecular mechanisms that regulate export of the planar cell-polarity protein Frizzled-6 out of the endoplasmic reticulum. J Biol Chem 10.1074/jbc.RA120.012835 (2020).
7. X. Tang, F. Yang, Y. Guo, Cell-free Reconstitution of the Packaging of Cargo Proteins into Vesicles at the trans Golgi Network. Bio Protoc 10, e3537 (2020).
8. S. J. Duguay et al., Post-translational processing of the insulin-like growth factor-2 precursor. Analysis of O-glycosylation and endoproteolysis. J Biol Chem 273, 18443-18451 (1998).
9. J. Dancourt, C. Barlowe, Protein sorting receptors in the early secretory pathway. Annu Rev Biochem 79, 777-802 (2010).
10. L. Lyu et al., Selective export of autotaxin from the endoplasmic reticulum. J Biol Chem 292, 7011-7022 (2017).
11. T. Buechling, V. Chaudhary, K. Spirohn, M. Weiss, M. Boutros, p24 proteins are required for secretion of Wnt ligands. EMBO Rep 12, 1265-1272 (2011).
12. X. Li et al., Drosophila p24 and Sec22 regulate Wingless trafficking in the early secretory pathway. Biochem Biophys Res Commun 463, 483-489 (2015).
13. C. Barlowe, A. Helenius, Cargo Capture and Bulk Flow in the Early Secretory Pathway. Annu Rev Cell Dev Biol 32, 197-222 (2016).
14. Y. Guo, D. W. Sirkis, R. Schekman, Protein sorting at the trans-Golgi network. Annu Rev Cell Dev Biol 30, 169-206 (2014).
15. S. Ouyang, B. Jia, W. Xie, J. Yang, Y. Lv, Mechanism underlying the regulation of sortilin expression and its trafficking function. J Cell Physiol 235, 8958-8971 (2020).
16. D. M. Conlon, Role of sortilin in lipid metabolism. Curr Opin Lipidol 30, 198-204 (2019).
17. S. Y. Xu, J. Jiang, A. Pan, C. Yan, X. X. Yan, Sortilin: a new player in dementia and Alzheimer-type neuropathology. Biochem Cell Biol 96, 491-497 (2018).
18. H. Talbot et al., Regulatory Roles of Sortilin and SorLA in Immune-Related Processes. Front Pharmacol 9, 1507 (2018).
19. M. Ariga, T. Nedachi, H. Katagiri, M. Kanzaki, Functional role of sortilin in myogenesis and development of insulin-responsive glucose transport system in C2C12 myocytes. J Biol Chem 283, 10208-10220 (2008).
20. T. M. Teslovich et al., Biological, clinical and population relevance of 95 loci for blood lipids. Nature 466, 707-713 (2010).
21. N. J. Samani et al., Genomewide association analysis of coronary artery disease. N Engl J Med 357, 443-453 (2007).
22. K. Musunuru et al., From noncoding variant to phenotype via SORT1 at the 1p13 cholesterol locus. Nature 466, 714-719 (2010).
23. M. Kjolby et al., Sort1, encoded by the cardiovascular risk locus 1p13.3, is a regulator of hepatic lipoprotein export. Cell Metab 12, 213-223 (2010).
24. E. Erbay, I. H. Park, P. D. Nuzzi, C. J. Schoenherr, J. Chen, IGF-II transcription in skeletal myogenesis is controlled by mTOR and nutrients. J Cell Biol 163, 931-936 (2003).
25. M. S. Yoon, J. Chen, PLD regulates myoblast differentiation through the mTOR-IGF2 pathway. J Cell Sci 121, 282-289 (2008).
26. A. Polesskaya et al., Lin-28 binds IGF-2 mRNA and participates in skeletal myogenesis by increasing translation efficiency. Genes Dev 21, 1125-1138 (2007).
27. X. Tang et al., A SURF4-to-proteoglycan relay mechanism that mediates the sorting and secretion of a tagged variant of sonic hedgehog. Proc Natl Acad Sci USA 119, e2113991119 (2022).
28. G. Boncompain et al., Synchronization of secretory protein traffic in populations of cells. Nat Methods 9, 493-498 (2012).
29. Y. Mao et al., The exocyst functions in niche cells to promote germline stem cell differentiation by directly controlling EGFR membrane trafficking. Development 146 (2019).
30. L. Niu et al., Atlastin-mediated membrane tethering is critical for cargo mobility and exit from the endoplasmic reticulum. Proc Natl Acad Sci USA 116, 14029-14038 (2019).
31. Y. Huang et al., An in vitro vesicle formation assay reveals cargo clients and factors that mediate vesicular trafficking. Proc Natl Acad Sci USA 118 (2021).
32. M. Marzioch et al., Erp1p and Erp2p, partners for Emp24p and Erv25p in a yeast p24 complex. Mol Biol Cell 10, 1923-1938 (1999).
33. D. Gommel et al., p24 and p23, the major transmembrane proteins of COPI-coated transport vesicles, form hetero-oligomeric complexes and cycle between the organelles of the early secretory pathway. FEBS Lett 447, 179-185 (1999).
34. J. Fullekrug et al., Localization and recycling of gp27 (hp24gamma3): complex formation with other p24 family members. Mol Biol Cell 10, 1939-1955 (1999).
35. N. Pastor-Cantizano, J. C. Montesinos, C. Bernat-Silvestre, M. J. Marcote, F. Aniento, p24 family proteins: key players in the regulation of trafficking along the secretory pathway. Protoplasma 253, 967-985 (2016).
36. M. Zhang et al., A Translocation Pathway for Vesicle-Mediated Unconventional Protein Secretion. Cell 181, 637-652 e615 (2020).
37. N. Nakamura et al., Identification of potential regulatory elements for the transport of Emp24p. Mol Biol Cell 9, 3493-3503 (1998).
38. R. Aber, W. Chan, S. Mugisha, L. A. Jerome-Majewska, Transmembrane emp24 domain proteins in development and disease. Genet Res (Camb) 101, e14 (2019).
39. V. Anantharaman, L. Aravind, The GOLD domain, a novel protein module involved in Golgi function and secretion. Genome Biol 3, research0023 (2002).
40. G. Huang et al., Insulin responsiveness of glucose transporter 4 in 3T3-L1 cells depends on the presence of sortilin. Mol Biol Cell 24, 3115-3122 (2013).
41. A. Denzel et al., The p24 family member p23 is required for early embryonic development. Curr Biol 10, 55-58 (2000).
42. S. Mitrovic, H. Ben-Tekaya, E. Koegler, J. Gruenberg, H. P. Hauri, The cargo receptors Surf4, endoplasmic reticulum-Golgi intermediate compartment (ERGIC)-53, and p25 are required to maintain the architecture of ERGIC and Golgi. Mol Biol Cell 19, 1976-1990 (2008).
43. J. H. Shin et al., Down-regulated TMED10 in Alzheimer disease induces autophagy via ATG4B activation. Autophagy 15, 1495-1505 (2019).
44. W. J. Belden, C. Barlowe, Erv25p, a component of COPII-coated vesicles, forms a complex with Emp24p that is required for efficient endoplasmic reticulum to Golgi transport. J Biol Chem 271, 26939-26946 (1996).
45. S. Takida, Y. Maeda, T. Kinoshita, Mammalian GPI-anchored proteins require p24 proteins for their efficient transport from the ER to the plasma membrane. Biochem J 409, 555-562 (2008).
46. T. Del Olmo et al., RAB21 interacts with TMED10 and modulates its localization and abundance. Biol Open 8 (2019).
47. C. Bonnon, M. W. Wendeler, J. P. Paccaud, H. P. Hauri, Selective export of human GPI-anchored proteins from the endoplasmic reticulum. J Cell Sci 123, 1705-1715 (2010).
48. A. Copic, C. F. Latham, M. A. Horlbeck, J. G. D'Arcangelo, E. A. Miller, ER cargo properties specify a requirement for COPII coat rigidity mediated by Sec13p. Science 335, 1359-1362 (2012).
49. J. G. D'Arcangelo et al., Traffic of p24 Proteins and COPII Coat Composition Mutually Influence Membrane Scaffolding. Curr Biol 25, 1296-1305 (2015).
50. M. Fujita et al., Sorting of GPI-anchored proteins into ER exit sites by p24 proteins is dependent on remodeled GPI. J Cell Biol 194, 61-75 (2011).
51. G. A. Castillon et al., The yeast p24 complex regulates GPI-anchored protein transport and quality control by monitoring anchor remodeling. Mol Biol Cell 22, 2924-2936 (2011).
52. F. X. Contreras et al., Molecular recognition of a single sphingolipid species by a protein's transmembrane domain. Nature 481, 525-529 (2012).
53. T. A. Nguyen, J. Debnath, Unconventional secretion: cargo channeling by TMED10. Cell Res 30, 713-714 (2020).
54. C. L. Evavold, J. C. Kagan, Diverse Control Mechanisms of the Interleukin-1 Cytokine Family. Front Cell Dev Biol 10, 910983 (2022).
55. A. J. Ping et al., Genetic linkage of Beckwith-Wiedemann syndrome to 11p15. Am J Hum Genet 44, 720-723 (1989).
56. A. Y. Kalebi, M. J. Hale, M. L. Wong, T. Hoffman, J. Murray, Surgically cured hypoglycemia secondary to pleural solitary fibrous tumour: case report and update review on the Doege-Potter syndrome. J Cardiothorac Surg 4, 45 (2009).
57. C. Gicquel et al., Epimutation of the telomeric imprinting center region on chromosome 11p15 in Silver-Russell syndrome. Nat Genet 37, 1003-1007 (2005).
58. T. Li, F. Yang, Y. Heng, Y. Guo, TMED10 mediates the trafficking of insulin-like growth factor 2 along the secretory pathway for myoblast differentiation PNAS 120 (46) e2215285120

We claim:

1. A pharmaceutical composition for inhibiting IGF2 signaling, the composition comprising an inhibitor of IGF2-TMED10 interaction in a cell and one or more pharmaceutical carriers or excipients.

2. The composition of claim 1, wherein the inhibitor comprises: (a) a human IGF2 polypeptide that comprises residues 112-140 of the human IGF2 sequence (SEQ ID NO:1); (b) a human TMED10 polypeptide that comprises residues 1-130 of the human TMED10 sequence; (c) a small molecule; (d) a covalent inhibitor; (e) an antibody; or (f) a genome editing tool.

3. A method of treating a subject having abnormal IGF2 signaling, the method comprising: (a) obtaining a pharmaceutical composition comprising an inhibitor of IGF2-TMED10 interaction in a cell and optionally one or more pharmaceutical carriers or excipients; and (b) administering an effective amount of the composition to the subject, wherein the inhibitor comprises a human IGF2 polypeptide comprising residues 112-140 of IGF2 sequence, a human TMED10 polypeptide that comprises residues 1-130 of the human TMED10 sequence, a small molecule, a covalent inhibitor, an antibody, or a genome editing tool.

4. The method of claim 3, wherein the subject is a mammal.

5. The method of claim 4, wherein the mammal is a human.

6. The method of claim 3, wherein the subject is affected by Beckwith-Wiedemann syndrome, Silver-Russell syndrome, or Doege-Potter syndrome.

7. The method of claim 3, wherein the secretion of IGF2 in the cell is blocked by the inhibitor.

8. A method of treating cancer in a subject in need thereof, the method comprising: (a) providing a pharmaceutical composition comprising an inhibitor of the IGF2-TMED10 interaction in a cell and optionally one or more pharmaceutical carriers or excipients; and (b) administering an effective amount of the composition to a subject affected by a cancer, and optionally one or more pharmaceutical carriers or excipients, wherein the inhibitor comprises a human IGF2 polypeptide comprising residues 112-140 of IGF2 sequence, a human TMED10 polypeptide that comprises residues 1-130 of the human TMED10 sequence, a small molecule, a covalent inhibitor, an antibody; or a genome editing tool.

9. The method of claim 8, wherein the subject is a mammal.

10. The method of claim 9, wherein the mammal is a human.

11. The method of claim 8, wherein the secretion of IGF2 in the cell is blocked by the inhibitor.

12. The method of claim 8, wherein the composition is administered to the subject via injection.

13. The method of claim 3, wherein the inhibitor comprises a CRISPR/CAS9 (RNA-guided targeted) genome editing tool, wherein the CRISPR/CAS9 editing tool is used to mutate the IGF2 sequence that codifies for residues 112-140 of IGF2.

14. The method of claim 3, wherein the inhibitor comprises a CRISPR/CAS9 (RNA-guided targeted) genome editing tool, wherein the CRISPR/CAS9 editing tool is used to mutate the human TMED10 sequence that codifies for residues 1-130 of the human TMED10 sequence, wherein the mutated TMED10 blocks the TMED10 binding to IGF2.

15. The method of claim 8, wherein the inhibitor comprises a CRISPR/CAS9 (RNA-guided targeted) genome editing tool, wherein the CRISPR/CAS9 editing tool is used to mutate the IGF2 sequence that codifies for residues 112-140 of IGF2.

16. The method of claim 8, wherein the inhibitor comprises a CRISPR/CAS9 (RNA-guided targeted) genome editing tool, wherein the CRISPR/CAS9 editing tool is used to mutate the human TMED10 sequence that codifies for residues 1-130 of the human TMED10 sequence, wherein the mutated TMED10 blocks the TMED10 binding to IGF2.
